(19) United States
(12) Patent Application Publication
(10) Pub. No.: US 2026/0151147 A1
(43) Pub. Date: Jun. 4, 2026

(54) ASPIRATION CLOT REMOVAL APPARATUSES AND METHODS

(71) Applicant: JUPITER ENDOVASCULAR INC., Menlo Park, CA (US)

(72) Inventors: Garrett J. Gomes, Pleasant Hill, CA (US); Jeff Haydon, San Mateo, CA (US); Alexander Q. Tilson, Burlingame, CA (US)

(21) Appl. No.: 19/405,388

(22) Filed: Dec. 2, 2025

Related U.S. Application Data
(60) Provisional application No. 63/728,150, filed on Dec. 4, 2024.

(51) Int. Cl. A61B 17/22; A61B 17/00; A61M 1/00

(52) U.S. Cl. CPC A61B 17/22; A61M 1/60; A61M 1/75; A61M 1/79; A61B 2017/00398; A61B 2017/00544; A61B 2017/00561; A61B 2017/00862; A61B 2017/00867; A61B 2017/22079; A61M 2202/0014; A61M 2202/0021; A61M 2202/0413; A61M 2202/06; A61M 2205/0216; A61M 2205/0266; A61M 2205/103; A61M 2205/3334; A61M 2205/3344; A61M 2205/50; A61M 2205/7545; A61M 2210/12

(57) ABSTRACT

Methods and apparatuses for blood clot aspiration may include the use of a positive pressure pump that is configured to provide high flow rates and/or high pressure upon activation. In particular, methods and apparatus using peristaltic pumps for providing on-demand and high flow rates are described herein. Also described herein are vacuum augmented peristaltic pumps and methods of using them, including methods of using them for clot removal.

### CROSS-REFERENCES TO RELATED APPLICATIONS

The present application claims the benefit of and priority to U.S. Provisional Patent Application No. 63/728,150, filed Dec. 4, 2024, the content of which is hereby incorporated by reference in its entity.

### INCORPORATION BY REFERENCE

All publications and patent applications mentioned in this specification are herein incorporated by reference in their entirety to the same extent as if each individual publication or patent application was specifically and individually indicated to be incorporated by reference.

### BACKGROUND

Clot extraction, such as the removal of thrombus material from vasculature, can be accomplished using various methods, including mechanical retrieval, pressurized fluid streams, and aspiration techniques. Aspiration systems are commonly employed and involve applying vacuum pressure along the lumen of a catheter. The vacuum is generated by a proximal low-pressure source, such as a vacuum pump, and directed toward a distal opening of the catheter. These systems often incorporate features like valves, gauges, disconnects, flush ports, guidewire access ports, and filters to streamline the process and facilitate blood re-infusion.

In more complex cases, clot extraction can involve additional challenges. For example, treating pulmonary embolisms may necessitate thrombolytic agents, which are costly, pose risks of severe complications, and are often ineffective for large or older clots. In severe cases, surgical intervention such as a sternotomy with bypass may be required, which is invasive, traumatic, and associated with extended hospital stays and recovery periods.

Advanced clot extraction techniques may include filtering the extracted blood to separate the clot material while returning the non-clotted blood to the patient's vasculature. This process minimizes blood loss and enables continuous blood flow during clot removal. Such simultaneous blood return and clot aspiration procedures often involve intricate steps that must be executed in a specific sequence.

There remains a critical need for improved methods and devices that enhance the safety and efficiency of blood clot aspiration, particularly by simplifying fluid management and optimizing clot removal processes.

### SUMMARY OF THE DISCLOSURE

Described herein are methods and apparatuses, including systems and devices, for cardiovascular treatments, and in particular for blood clot aspiration procedures. For example, described herein are various systems and components configured for safe extraction of clot material and improved fluid management during clot aspiration procedures. In particular, these methods and apparatuses may include one or more peristaltic pump apparatuses. These peristaltic pump apparatuses may be configured to permit relatively high flow rates (e.g., 1.0 L/min or more, 1.5 L/min or more, 2.0 L/min or more, 2.5 L/min or more, 3.0 L/min or more, 3.5 L/min or more, 4.0 L/min or more, 4.5 L/min or more, 5.0 L/min or more, 5.5 L/min or more, 6 L/min or more, 10 L/min or more, 20 L/min or more, etc.). The pump apparatuses described herein may be operated in a continuous mode, and/or an intermittent mode, and/or a pulsed mode, and/or an on-demand mode, or any combination of these modes. For example, a pump apparatus may be operated in a continuous mode at a low flow rate for a first use and in a burst on-demand mode for a second use.

Any of the methods and apparatuses (e.g., devices, systems, assemblies, sub-assemblies, etc.) described herein may be configured to provide return of filtered blood that has been removed from the body back to the body. The apparatuses may include one or more components for filtering the clot material from aspirated blood and return filtered blood back into the patient. The systems may include features that allow for easier management of the blood and other fluids (e.g., air, vacuum) during clot aspiration procedures, thereby allowing for safer overall procedures.

The methods and apparatuses described herein may be used in blood clot aspiration procedures performed in a vasculature of a subject. As used herein, vasculature may include any vascular region of the body, including, but not limited to, regions of the heart, arteries, veins, capillaries, peripheral vasculature, and neurovascular structures. In general, the methods and apparatuses described herein are particularly useful for vascular indications, including cardiovascular, peripheral vascular, cerebrovascular, neurovascular, pulmonary vascular, thoracic vascular, abdominal vascular, lymphatic vascular, renal vascular, and/or genitourinary vascular indications.

The apparatuses may include components configured to aspirate the blood clot material from the patient. For example, an aspiration catheter may include an elongate distal catheter portion that is adapted to be inserted and advanced through a patient's vasculature toward the clot material. The aspiration catheter may include a control that a user can use to control the suction applied within the vasculature. Such control may be on a handle at a proximal end of the aspiration catheter or may be separate from the aspiration catheter. The control may be coupled (directly or indirectly, e.g., wirelessly) to the pump (e.g., the peristaltic pump apparatus/assembly).

In general, the methods and apparatuses described herein may operate without the need for stored vacuum, by providing on-demand aspiration. This may be particularly beneficial as it may avoid the exposure blood to vacuum for extended periods and may prevent strain on the system. The peristaltic pump apparatuses described herein may also be effectively valveless; the peristaltic pump may control the flow through the continuous fluid path, without requiring additional valves during operation.

The apparatuses may include components that manage blood once it is removed from the patient's body. In some examples, the components may be adapted to filter clot material out of the blood and return the filtered blood back into the patient. The components may be connected by lines (e.g., tubing) configured to provide the flow of fluid (e.g., blood, air, vacuum) to and from the various components. Such components and lines may collectively form a blood return circuit for returning the aspirated blood to the patient.

The apparatuses may include an aspiration catheter that is configured to aspirate blood, including clot material, from a patient's vasculature. A clot capture chamber may be configured to receive blood from an aspiration catheter and capture and contain at least some of the clot material from the aspirated blood. The clot capture chamber may be made of a clear or transparent material, or may include a clear portion (e.g., window) or portions to allow contents of the clot capture chamber to be viewed from outside the clot capture chamber. The clot capture chamber may include features that allow a user (e.g., doctor or assistant) to access the clot material and to easily handle the clot capture chamber. For example, the clot capture chamber may include a clot capture vent valve that is configured to allow venting of the clot capture chamber to an external atmosphere. The clot capture chamber may include a lid that is configured to automatically open, for example, when the clot capture chamber is vented. This allows the user to easily access a removable tray that captures the clot material, for example to access the tray for analysis of the clot material or to empty the tray. The clot capture chamber may include a latch that retains the lid in a closed position before and after accessing the tray. The removable tray may have a perforated surface that is sloped to help reduce clogging and provide efficient flow of blood through the clot capture chamber. The clot capture chamber may include a valve that fluidly disconnects the clot capture chamber from the aspiration catheter, such as to prevent inadvertent blood loss during a procedure. This valve may be actuatable separately or as part of the same control interface as unlatching or opening the lid of the clot capture.

The apparatuses described herein may include a first reservoir that is configured to receive blood from the clot capture chamber, and/or to receive unfiltered blood, and may include a filter that is configured to filter the blood. The first reservoir may be made of a clear material, or may include a clear portion (e.g., window) or portions that allow a user to visualize a level of filtered blood contained within the reservoir. In some cases, the first reservoir includes graduation marks on or near a clear portion so that a user can easily determine a present amount of blood stored within the reservoir.

The apparatuses may include a blood return syringe that is configured to receive filtered blood from the first reservoir or another reservoir and to inject the filtered blood into the patient's vasculature. The blood return syringe may include a chamber (e.g., barrel) and a plunger that is used to pull the filtered blood into the chamber and to push the filtered blood into the patient. The syringe may be connected to filtered blood and the patient's vasculature via one-way valves to prevent backflow into the filtered blood and to prevent aspiration of blood from the patient. The syringe may be connected to filtered blood and the patient's vasculature via user controllable or electrically activated flow control valves.

The apparatuses described herein may include a second reservoir to collect filtered blood. The second reservoir may be either separate from or integral with the first reservoir configured to receive blood from the clot chamber. If integral, one or more walls and/or one or more filters may separate the second reservoir from the first reservoir, for example to separate unfiltered blood from filtered blood, or to filter unfiltered blood in the first reservoir before it enters the second reservoir. This second reservoir may be connected to the patient's vasculature via tubing and filters to return the filtered blood. This second reservoir may have a vent to the atmosphere in the upper portion of the chamber. The vent may have a filter to prevent ingress of bacteria and other harmful materials into the second reservoir. The vent allows blood to be returned to the patient via a pressure differential generated by the atmosphere (e.g., with the second reservoir at a selected height) and pressure of the patient's vasculature. The second reservoir may be raised in height to increase this differential and thus increase the flow rate of the blood return. Alternatively, this vent can be connected to a source of positive pressure such as a syringe or bulb inflator. The user can increase the pressure within the second reservoir to force blood back into the patient at a desired target pressure and flow rate. The second reservoir may have a valve on the vent that remains closed when the second reservoir is at a pressure below a desired level (relative to atmospheric pressure) and opens above that pressure to allow air egress to maintain the desired pressure within the second reservoir. This pressure can be user selected or manufactured into the device, for example with a relief pressure of 100 mmHg or 200 mmHg or 300 mmHg to prevent excessive pressure from damaging the blood or causing harm to the patient.

Filtered blood may be reinfused into the patient's vasculature throughout the procedure with or without user interaction. For example, the operator can continue to operate the aspiration system while the reinfusion chamber reinfuses blood back into the patient via gravity feed or pressure control as described herein. In some case the reinfusion (e.g., collection) chamber may be pressurized or pressurizable, which may assist with the return of blood, e.g., adding positive pressure to drive flow from the chamber into the body.

Any of the apparatuses described herein may include a rigidizing aspiration sheath catheter. The rigidizing aspiration sheath catheter may be advanced within the subject's vasculature toward a target location (e.g., that includes a blood clot). The rigidizing aspiration sheath catheter may be advanced within the vasculature while in a flexible state, then rigidized into a rigid state once at the target location. The clot material may be aspirated through the rigidizing aspiration sheath catheter in the rigid state to remove the clot material from the vasculature. In some examples, an aspiration catheter may be inserted and advanced through the rigidizing aspiration sheath catheter to aspirate the clot material using the aspiration catheter. The rigidizing aspiration sheath catheter may be configured to apply aspiration (e.g., suction) directly through the lumen of the rigidizing aspiration sheath catheter and/or may be configured to receive an aspiration catheter (which, in some examples, may or may not also be rigidizing).

A rigidizing aspiration sheath catheter may be rigidized by the application of pressure (negative and/or positive) within walls of the rigidizing aspiration sheath catheter. In some examples, a same suction (vacuum) line may be attached (swapped between) the aspiration catheter and the rigidizing aspiration sheath catheter. Alternatively in some examples, suction may be applied to both the rigidizing aspiration sheath catheter and an aspiration catheter within the lumen of the rigidizing aspiration sheath catheter (e.g., through the aspiration catheter and/or around the aspiration catheter).

Also described herein are aspiration catheters (which may be rigidizing or non-rigidizing), a suction line, an in-line vacuum activation valve, and a clot capture chamber. All of some of these components may be used together, e.g., as part of system, or each of them may be used separately and may be configured to include elements of particular use in removing clot material.

The apparatuses and methods described herein may be used for aspiration of clot material from any region of the vasculature, including (but not limited to) pulmonary embolism, peripheral (e.g., arterial embolism), central (e.g., cerebral thrombus), etc., including treatment of stroke.

The aspiration of clots can involve removal of blood. The blood must be captured, and in some cases the blood can be returned to the patient. The present invention contemplates methods to capture, store and/or return blood to the patient. These include the use of filters and containers that are capable of separating blood, which may include separating blood in a sterile field. A system may also anticipate the use of standard cardiotomy reservoirs. Cardiotomy reservoirs are indicated for use in cardiopulmonary bypass circuits during surgery. Intended uses include an air-fluid separation chamber, a temporary storage reservoir for priming solutions, filtration of particulate materials (clots, blood cell aggregates, etc.). Design accommodations would allow these reservoirs to be uniquely adapted to an aspiration embolectomy system.

One or more of components of the apparatuses described herein may be configured to remain within the sterile field during a medical procedure while other components of the system may be configured to remain outside of the sterile field during the medical procedure. In cases where a rigidizing aspiration sheath catheter is used, the rigidizing aspiration sheath catheter may be positioned within the body and used for multiple introductions and removal steps, which may in some cases be performed without requiring the use of a guidewire. Aspiration without a guidewire enables larger luminal area (and therefore greater suction), reduces guidewire risks, and enables smoother clot transit, because the clot does not need to shear relative to the wire. This may allow the system to be operated within the limited sterile field during the entire procedure.

Any of the aspiration catheters and/or rigidizing aspiration sheath catheters described herein may include an atraumatic tip at the distal end of the aspiration catheter or rigidizing aspiration sheath catheter. For example, the distal end (tip) region may be formed of a relatively soft (e.g., low durometer) material and may be rounded.

A vacuum line for the aspiration catheter may include: a mating attachment at a distal end of vacuum line configured to lockingly engage with the mating attachment connector of the aspiration catheter. As mentioned, the mating attachment may be a universal attachment for coupling with one or more of the catheters of a system. For example, the mating attachment may include a bayonet-type attachment as well as one or more finger-grip regions enhancing the ease of use. The vacuum line may include vacuum tubing. Any of these apparatuses may include a vacuum activation valve. An apparatus may be hand-triggered, comprising a handle and configured to open or close the vacuum line, wherein the hand-triggered vacuum activation valve is connected in-line with the mating attachment, and a proximal end of the vacuum line is further configured to couple to a vacuum pump and a blood collection chamber. This valve may be a roller mechanism that pinches the vacuum tubing locally.

In some examples the apparatuses include a clot capture chamber that may be connected in-line with a hand-triggered vacuum activation valve and may include a visualization chamber mounted above an exit port, wherein the exit port is connected to the proximal end region of the vacuum line. The clot capture chamber may include a removable tray within the visualization chamber. The removable tray may be sized to conform to an inner perimeter of the clot capture container. In some examples the clot capture chamber comprises a transparent lid, which may include one or more tabs. The tray may have a mesh size such that blood passes through, but clot does not. For example, the mesh may have a mesh size of between about 0.037 mm and about 6 mm (e.g., between about 0.05 mm and about 5 mm, between about 0.1 mm and about 4 mm, between about 0.2 mm and 3 mm, between about 0.3 mm and 2.5 mm, between about 0.5 mm and about 4 mm, between about 0.75 mm and about 4 mm, etc.).

As mentioned, any of these apparatuses may include an aspiration catheter configured to be inserted through a central bore of, and into a lumen of, a rigidizing aspiration sheath catheter, the aspiration catheter comprising a second mating attachment connector, wherein the mating attachment at the distal end of vacuum line is configured to lockingly engage with the second mating attachment connector or the first mating attachment connector. The aspiration catheter may comprise an aspiration lumen extending therethrough and an aspiration catheter hemostasis valve region at a proximal end.

For example, described herein are apparatuses, for removing a clot material, comprising: an aspiration line configured to couple to an aspiration catheter; a clot capture chamber in fluid communication with the aspiration line and configured to capture the clot material; a peristaltic pump apparatus coupled to the aspiration line; a collection chamber coupled to the aspiration line and configured to collect blood aspirated through the aspiration line; and a control for controlling operation of the peristaltic pump apparatus, wherein the control is configured to cause the peristaltic pump apparatus to apply aspiration to the aspiration line at a high flow rate when activated.

A peristaltic pump apparatus may be configured to apply aspiration at a high flow rate of, e.g., 1.0 L/min or more, 1.25 L/min or more, 1.5 L/min or more, 1.75 L/min or more, 2 L/min or more, 2.25 L/min or more, 2.5 L/min or more, 2.75 L/min or more, 3 L/min or more, 3.25 L/min or more, 3.5 L/min or more, 3.75 L/min or more, 4 L/min or more, 4.5 L/min or more, 5 L/min or more, 5.5 L/min or more, 6 L/min or more, 6.5 L/min or more, 7 L/min or more, 7.5 L/min or more, 8 L/min or more, 8.5 L/min or more, 9 L/min or more, etc. In some examples the peristaltic pump apparatus may be configured to apply aspiration at a high flow rate of 5 L/min or greater when activated by the control.

Any of these peristaltic pump apparatuses may be configured to deliver a high flow rate and/or to automatically allow either a high flow rate/low pressure or a high pressure/low flow rate. For example, the peristaltic pump apparatus may comprise two or more peristaltic pumps arranged in parallel; in some cases the two or more peristaltic pumps arranged in parallel comprise a first pump having a high flow and low pressure and a second pump having a low flow and high pressure. Alternatively or additionally, the peristaltic pump apparatus may be a vacuum augmented peristaltic pump, in which an outside of the tubing being driven by the peristaltic pump to generate the pressure/flow is held under pressure rather than ambient pressure. In particular, the outside tubing may be maintained under a negative pressure/vacuum. In any of these examples, the two or more peristaltic pumps may be arranged in parallel both have low flow and high pressure; in some cases pressure is not additive but the flow rate may be summed, to achieve a high flow by having more than one pump in parallel, as described herein.

The methods and apparatuses described herein including the peristaltic pumps may achieve a vacuum pressure of, for example-715 mmHg,-750 mmHg. Such negative pressures may be even higher magnitude than other types of pumps, including ‘high powered’ pumps (e.g., which may be rated for-713 mmHg). Although syringes can achieve close to-760 mmHg (theoretical max vacuum) they are not capable of continuous flow. The methods and apparatuses described herein may achieve continuous flow aspiration at greater than-750 mmHg and/or applying simultaneous negative pressure and positive pressure wherein the negative pressure is greater than about-750 mmHg.

In any of these apparatuses, a collection chamber may be part of a blood return sub-system further comprising a blood return line configured to couple to a blood return inlet on the patient. For example, the blood return sub-system may comprise a driving pump configured to drive fluid into the patient. The collection chamber may comprise an expandable container. These apparatuses may include one or more filters to remove clot material. For example, the collection chamber may comprise one or more filters.

In general, these apparatuses may be configured to minimize blood loss, such as by including one or more valves that are configured to prevent flow through the aspiration line when the pump is not on. For example, the apparatus may be configured to prevent flow through the aspiration line when the peristaltic pump apparatus is not activated and/or aspiration is not being applied through the aspiration catheter.

In any of these apparatuses, a clot capture container may be coupled to an aspiration line proximal to a peristaltic pump apparatus or coupled distal to the peristaltic pump apparatus. The collection chamber may be coupled to the aspiration line distal to the peristaltic pump apparatus.

A control of a pump may be configured to apply aspiration at a first, high, flow rate for a first time, then at a second, lower flow rate for a second time. Alternatively, the control may be configured to apply aspiration at a first, low, flow rate for a first time, then at a second, higher flow rate for a second time. Switching between the first flow rate and the second flow rate may occur upon user input to the control, or automatically (e.g., based on input to the control from a sensor.

For example, a system for removing a clot material may include: an aspiration line configured to couple to an aspiration catheter; a clot capture chamber comprising a viewing window in fluid communication with the aspiration line and configured to capture the clot material for viewing through the viewing window; a peristaltic pump apparatus coupled to the aspiration line; a collection chamber coupled to the aspiration line and configured to collect blood aspirated through the aspiration line; and a control for controlling operation of the peristaltic pump, wherein the control is configured to cause the peristaltic pump apparatus to apply aspiration to the aspiration line when activated, further wherein the control is configured to open a valve in fluid communication with the aspiration line when activated and to close the valve when the peristaltic pump apparatus is not activated.

Also described herein are vacuum augmented peristaltic pumps. For example, a vacuum augmented peristaltic pump may include: a plurality of rollers configured to engage and compress a tubing; a rotor coupled to the plurality of rollers; a pressurized chamber configured to sealingly enclose the plurality of rollers and the tubing; a base comprising a motor that is configured to drive the rotor; and a vacuum inlet into the pressurized chamber configured to receive negative pressure from a vacuum source in order to generate a negative pressure within the pressurized chamber. Any of these pumps may include the tubing. The tubing may comprise a thin-walled tubing. In some cases, the thin-walled tubing may have a wall resiliency that is insufficient to fully expand the tubing without the application of negative pressure applied external to the tubing (e.g., internal to the pressurized chamber).

The pressurized chamber may include one or more seals to prevent leakage around the tubing and/or rotor. For example, any of these apparatuses may include a first tubing seal in the pressurized chamber and a second tubing seal in the pressurized chamber, wherein the first and second tubing seals are configured to pass the tubing through a wall of the pressurized chamber without substantially releasing the negative pressure within the pressurized chamber. The pressurized chamber may be configured to enclose the base. In some examples, the pressurized chamber does not enclose the base. Any of these apparatuses may include a rotor seal configured to prevent substantially releasing the negative pressure within the pressurized chamber around the rotor.

In some cases, the vacuum augmented peristaltic pump is configured to use a cartridge (e.g., a disposable/replaceable/swappable cartridge) that may include a portion of the pump, such as the pressurized chamber, and enclosing the tubing and/or the rollers. Optionally the cartridge may include a rotor or a portion of the rotor that may engage with the driver/motor (or a second portion of the rotor/driver) to drive rotation of the rollers and activation of the pump. The cartridge may be configured to couple to a source of negative pressure. Thus, the pressurized chamber and rollers may form a cartridge that is configured to be removably coupled to the base.

Any of these apparatuses may include a controller configured to receive input to actuate the motor. The controller may include hardware, software and/or firmware for controlling operation of the pump. The controller may include a microcontroller, timing circuitry, memory, one or more processors, etc. The controller may be configured to drive rollers in the pump at a first rate for a first time period to generate a first flow rate in the tubing, and at a second rate for a second period of time to generate a second flow rate in the tubing, wherein the second flow rate may be different than the first flow rate. In any of these examples, the controller may be configured to apply aspiration at first, high, flow rate for a first time, then at a second lower flow rate if clot is detected. Any of these apparatuses may be configured to shut off flow if clot is not detected or it has cleared the tubing. Any appropriate technique may be used to detect clot.

Any of these apparatuses may optionally include a vacuum source. The vacuum source may be a pump, syringe, wall-line source of vacuum, etc. The apparatus may include one or more sensors to regulate the applied pressure (e.g., via a feedback loop, e.g., implemented by the controller). The vacuum source may comprise a suction pump.

Any of these apparatuses may include a vacuum release configured to release all or some of the vacuum within the pressurized chamber, either to remove the cartridge and/or tubing, or to regulate the pressure within the vacuum chamber.

Any of these apparatuses may include a compression surface against which the tubing is compressed by one or more roller.

For example, a vacuum augmented peristaltic pump may include: a tubing; a plurality of rollers engaged with the tubing and configured to compress the tubing; a rotor coupled to the plurality of rollers; a pressurized chamber configured to sealingly enclose the plurality of rollers and the tubing; a motor configured to drive the rotor; and a vacuum source configured to generate a negative pressure within the pressurized chamber.

In any of these apparatuses and methods including VAPP, the vacuum source may be configured so that the peristaltic pump pumps itself down (e.g., forms a vacuum around the tubing and/or rollers. In any of these apparatuses and methods, the vacuum source may be another pump running off the same drive shaft. In any of these apparatuses and methods, the pressurizing chamber may be at least partially filled with a non-compressible fluid prior to the vacuum being applied, which may make pumping down faster. In any of these apparatuses and methods, a check valve may be included to allow a single peristaltic pump to also draw air out of its chamber without risk of back flow into the fluid path and without having to pump down again after use.

Also described herein are methods and apparatuses for removing a clot material that may include a vacuum augmented peristaltic pump. For example, a system may include: an aspiration line configured to couple to an aspiration catheter; and a vacuum augmented peristaltic pump comprising a plurality of rollers engaged with a length of peristaltic tubing in fluid communication with the aspiration line and configured to compress the tubing, a rotor coupled to the plurality of rollers, and a motor configured to drive the rotor, wherein the plurality of rollers and the length of peristaltic tubing is sealingly enclosed within a pressurized chamber configured to maintain a negative pressure around the length of peristaltic tubing.

Any of these apparatuses may include a vacuum source configured to generate a negative pressure within the pressurized chamber and/or a control for controlling operation of the vacuum augmented peristaltic pump, wherein the control is configured to cause the vacuum augmented peristaltic pump to apply aspiration to the aspiration line when activated. The controller may be configured to maintain the negative pressure within the pressurized chamber.

The apparatuses may include a clot capture chamber in fluid communication with the aspiration line and configured to capture clot material. For example, the clot capture chamber may be coupled to the aspiration line proximally to the vacuum augmented peristaltic pump. The clot capture chamber may be coupled to the aspiration line distal to the peristaltic pump apparatus.

Any of these apparatuses may include a collection chamber coupled to the aspiration line and configured to collect blood aspirated through the aspiration line. The collection chamber may comprise an expandable container. The collection chamber may comprise one or more filters.

The vacuum augmented peristaltic pump may be configured to apply aspiration at a high flow rate of 1.5 L/min or greater (e.g., 2 L/min or more, 3 L/min or more, 4 L/min or more, etc.). For example, the vacuum augmented peristaltic pump may be configured to apply aspiration at a high flow rate of 5 L/min or greater when activated. In any of these apparatuses, these pumps may be described in terms of the percentage (%) of a theoretical maximum flow for a given tube set. For example, for both small and large bore catheters under a “no clot” load, the pump may be able to achieve a % of full vacuum. For example, the pressure drop from the tip of the catheter to the pump, without a clot present, may be a percentage of 15 psi.

Any of these apparatuses may include a blood return sub-system configured to couple to a blood return inlet on the patient. For example, the blood return sub-system may comprise a second pump configured to drive fluid into the patient. The blood-return sub-system may be configured to drive blood through the blood return inlet using the vacuum augmented peristaltic pump. The system may include a control (e.g., button, switch, lever, pedal, slider, touchscreen, etc.) configured to activate the aspiration. The control may be further configured to open a valve on an aspiration line when the peristaltic pump apparatus is activated.

Also described herein are methods for the removing clot material from a vasculature of a patient, a method comprising: positioning a catheter at least partially within the vasculature proximate to the clot material, wherein the catheter is configured to be coupled to a peristaltic pump apparatus (e.g., fluidically coupled, so that fluid may be passed from the catheter to the pump and beyond, such as to a container, and/or re-introduced into the patient), and wherein activating the peristaltic pump apparatus fluidically connects the peristaltic pump apparatus to the catheter and wherein de-activating the peristaltic pump apparatus fluidically disconnects the peristaltic pump apparatus from the catheter; activating the peristaltic pump apparatus to generate negative pressure within the catheter to apply the negative pressure to generate a flow rate within the catheter of greater than about 5 L/min to aspirate at least a portion of the clot material into the catheter. The peristaltic pump apparatus may comprise a vacuum augmented peristaltic pump, and further comprising generating a negative pressure around an outside of the tubing of the vacuum augmented peristaltic pump prior to activating the peristaltic pump apparatus. In some cases, the peristaltic pump apparatus may comprise two or more peristaltic pumps arranged in parallel. For example, the two or more peristaltic pumps arranged in parallel may comprise a first pump having a high flow and low pressure and a second pump having a low flow and high pressure.

Also described herein are methods for the treatment of clot material within a vasculature of a patient. For example, a method may include: positioning a first catheter at least partially within the vasculature proximate to the clot material; applying a negative pressure to an outside of a length of peristaltic pump tubing of a peristaltic pump, wherein an inner lumen of the length of peristaltic pump tubing is in fluid communication with the first catheter; and activating the peristaltic pump to apply aspiration to aspirate at least a portion of the clot material and blood through the first catheter and into a clot capture chamber fluidically coupled to the first catheter, wherein the negative pressure applied to the outside of the peristaltic pump tubing is maintained while activating the peristaltic pump. As used herein, “fluidically coupled” may refer to connecting, including via tubing, so that fluid may flow between the two or more fluidically coupled components or regions, which do not need to be directly connected. In some cases, unless specified otherwise, the fluid connection between fluidically connected components, such as tubing, may be valved or metered.

Any of these methods may include collecting and filtering the blood removed from the patient and returning the filtered blood. For example, the method may include driving the blood return using the peristaltic pump. Returning the filtered blood may comprise returning the filtered blood through a second catheter into the vasculature.

Any of these methods may include observing a clot material aspirated through the aspiration catheter within a window of a clot capture chamber connected in-line with a hand-triggered vacuum activation valve.

Any of these apparatuses may be used with or without a guidewire, either for the entire procedure or for the portion of the procedure following initially placing the rigidizing aspiration sheath catheter (e.g., rigidizing overtube). For example, advancing the rigidizing aspiration sheath catheter may include advancing the rigidizing aspiration sheath catheter without the use of a guidewire. In some examples advancing the rigidizing aspiration sheath catheter comprises advancing the aspiration catheter distally relative to the rigidizing aspiration sheath catheter in the rigid state and steering a distal end of the aspiration catheter. The method may further include advancing the rigidizing aspiration sheath catheter in the flexible state over the aspiration catheter and rigidizing the rigidizing aspiration sheath catheter. In some examples advancing the rigidizing aspiration sheath catheter comprises advancing the aspiration sheath catheter with an obturator within a lumen of the rigidizing aspiration sheath catheter.

Any of these apparatuses may be systems that include one or more aspiration catheters. The aspiration catheter may include an integrated hemostasis region (e.g., at a proximal end). The proximal ends of the rigidizing aspiration sheath catheters and the aspiration catheters may be adapted to interchangeably engage with a suction (aspiration) line. Any of the rigidizing aspiration sheath catheters may be steerable or may be steered by using an obturator and/or guidewire. In some cases, a rigidizing aspiration sheath catheter includes one or more distal steering regions that may be steered from the proximal end, e.g., by pulling on a tendon. In some cases, an obturator configured to fit into the rigidizing aspiration sheath catheter may be steerable. In some cases, an obturator adapted to fit snugly into the rigidizing aspiration sheath catheter may adapt the rigidizing aspiration sheath catheter for use with a guidewire (e.g., the obturator may include a guidewire lumen).

All of the methods and apparatuses described herein, in any combination, are herein contemplated and can be used to achieve the benefits as described herein.

### BRIEF DESCRIPTION OF THE DRAWINGS

A better understanding of the features and advantages of the methods and apparatuses described herein will be obtained by reference to the following detailed description that sets forth illustrative embodiments, and the accompanying drawings of which:

FIG. 1A shows one example of a system including a rigidizing aspiration sheath catheter apparatus.

FIGS. 1B-1G show examples of a system and components of these systems.

FIGS. 2A-2B shows an example of a vacuum activation valve configured as a handle.

FIGS. 2C-2E illustrate an example of a vacuum activation valve configured as a handle.

FIGS. 3A-3H show various views of an example of a clot capture chamber.

FIGS. 4A and 4B show a traditional catheter and an example of a rigidizing catheter, respectively, navigating to a pulmonary artery.

FIGS. 5A-5B illustrate an example of a system including a rigidizing aspiration sheath catheter configured with a blood return circuit, including a blood filter and blood bag.

FIGS. 6A-6B illustrate an example of a system including a rigidizing aspiration sheath catheter configured with a blood return circuit without a blood bag.

FIGS. 7A-7B illustrate an example of a system including a rigidizing aspiration sheath catheter configured with a blood return circuit having separate access and return sites on the body.

FIGS. 8A-8B illustrate an example of a system including a rigidizing aspiration sheath catheter configured with a blood return circuit including a blood bag.

FIGS. 9A-9B illustrate an example of a system including a rigidizing aspiration sheath catheter including a blood return circuit using a syringe, as described herein.

FIG. 9C is an example of a portion of a tubing assembly that may be used with a blood return circuit.

FIGS. 10A-10B illustrate an example of a system including a rigidizing aspiration sheath catheter. FIG. 10A shows a schematic illustration of the system. FIG. 10B illustrates the system of FIG. 10A including an optional blood return circuit portion.

FIGS. 11A-11B illustrate an example of a clot capture chamber that is configured to reduce hemolysis.

FIGS. 12A-12B illustrate an example of a system including a venting control (e.g., venting button) that may reduce hemolysis.

FIGS. 13A-13G schematically illustrate examples of positive displacement pumps that may be used with any of these methods and apparatuses.

FIG. 14A schematically illustrates one example of a system for clot removal including a peristaltic pump apparatus including two or more parallel peristaltic pumps.

FIG. 14B schematically illustrates an example of a system for clot removal including a peristaltic pump apparatus including two or more parallel peristaltic pump heads.

FIGS. 14C and 14D show peristaltic pump rotors that are in-phase (FIG. 14A) and out-of-phase (FIG. 14B).

FIGS. 15A-15B illustrate examples of vacuum augmented peristaltic pumps.

FIGS. 15C-15E illustrate an example of a tubing that may be used as part of a peristaltic pump apparatus as described herein.

FIGS. 16A-16E schematically illustrate examples of systems for clot removal using a peristaltic pump apparatus.

FIGS. 17A-17B illustrate examples of systems for removing clot material using on-demand, positive displacement pumps, such as any of the peristaltic pump apparatuses described herein. FIG. 17A shows an example of a system for removing clot. FIG. 17B shows an example of a system for removing clot including blood return.

FIGS. 18A-18C illustrate examples of systems for removing clot material using on-demand, positive displacement pumps, such as any of the peristaltic pump apparatuses that include blood return as part of the clot removal circuit.

FIG. 18D schematically illustrates another example of a system for removing clot material using a peristaltic pump apparatus, including blood return via a gravity-fed technique.

FIGS. 19A-19B illustrate examples of systems for removing clot material using continuous vacuum pump systems. FIG. 19A shows an example of a system for removing clot.

FIG. 19B shows an example of a system for removing clot including blood return.

FIGS. 20A-20B illustrate examples of systems for removing clot material using continuous vacuum pump systems that include blood return as part of the clot removal circuit.

FIGS. 21A-21C illustrate a method of performing a pulmonary embolectomy using a rigidizing apparatus as described herein to form an “endoportal.”

FIGS. 22A-22B show a prior art technique for performing a pulmonary embolectomy.

FIGS. 23A-23D illustrate an enlarged view of the removal of clot material from a right pulmonary artery as part of a pulmonary embolectomy similar to that shown in FIGS. 21A-21C in which aspiration is applied through the rigidizing catheter.

FIGS. 24A-24E illustrate an enlarged view of the removal of clot material from a right pulmonary artery as part of a pulmonary embolectomy similar to that shown in FIGS. 21A-21C in which an inner aspiration catheter is used.

FIGS. 25A-25E illustrate an enlarged view of the removal of clot material from a left pulmonary artery as described herein using the rigidizing catheter and/or aspiration catheter shown in FIGS. 21A-21C and 24A-24E.

FIGS. 26A-26D illustrate the removal of a clot material from the left pulmonary artery using the rigidizing catheter and/or aspiration catheter shown in FIGS. 21A-2C and 24A-24E.

### DETAILED DESCRIPTION

Described herein are methods and apparatuses (e.g., devices, systems, assemblies, etc.) for vascular (e.g., cardiovascular) treatment. These methods and apparatuses may be configured for removal of clot material (e.g., for clot capture) by aspiration. The systems may include components for aspirating blood clot material from a patient's body, filtering the blood from the clot material, and returning the filtered blood back into the patient's body. The components that filter and return the blood to the patient may be referred to as a blood return circuit. The systems may include features that provide real-time control and visualization of various components used during a clot aspiration procedure, including the blood return circuit, thereby providing a safer overall procedure.

In particular, these methods and apparatuses (e.g., devices, systems, etc.) may include a positive displacement pump assembly, and in particular a peristaltic pump assembly. These peristaltic pump assemblies may be configured to provide very rapid (e.g., time to high pressure and/or high flow rate) and/or high flow rate and/or high pressure peristaltic pumps. In some examples the peristaltic pump assembly includes a plurality of, e.g., two or more, peristaltic pumps in parallel. Note that in the description herein, where two or more peristaltic pumps are described it should be understood that this may refer to two or more peristaltic pump heads (e.g., sets of rollers or other compression members) that may be part of the same assembly, e.g., may share a single motor and/or rotor, and may be a part of the same housing; in some cases each of the two or more peristaltic pumps may be separate peristaltic pumps that may have separate motors and/or rotors for driving the pump heads. Even where separate motors are used, the same controller may control and/or coordinate, the rotation of the peristaltic heads. In apparatuses having a plurality of pump heads, the phase of the rollers of the two heads may be configured to facilitate clot engagement or transit down an aspiration catheter. Any of the peristaltic pump assemblies described herein may be a vacuum augmented peristaltic pump.

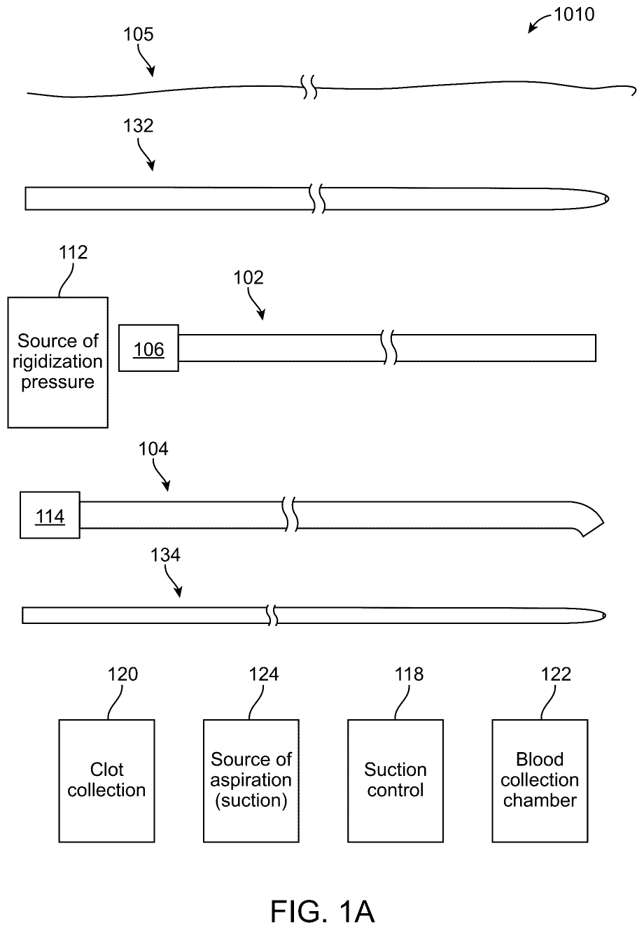

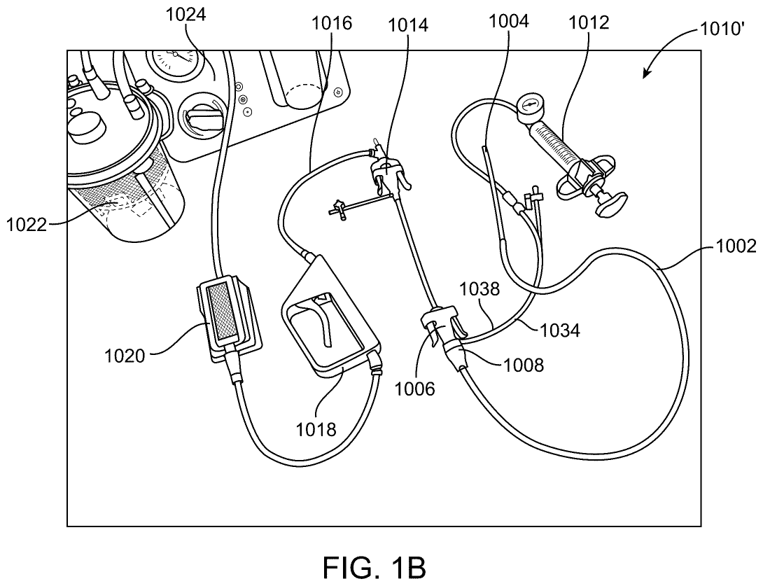

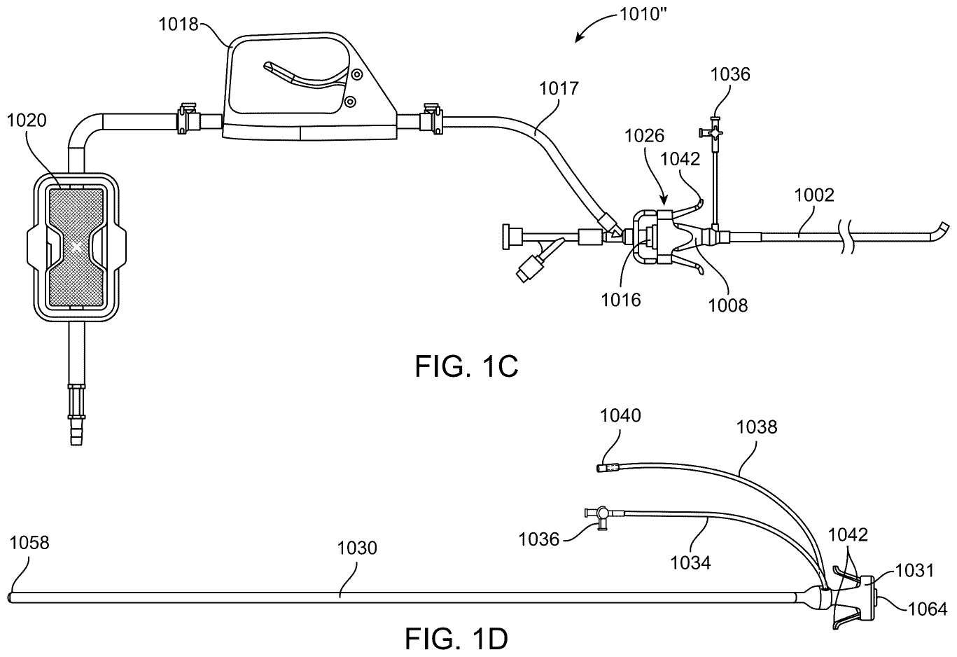

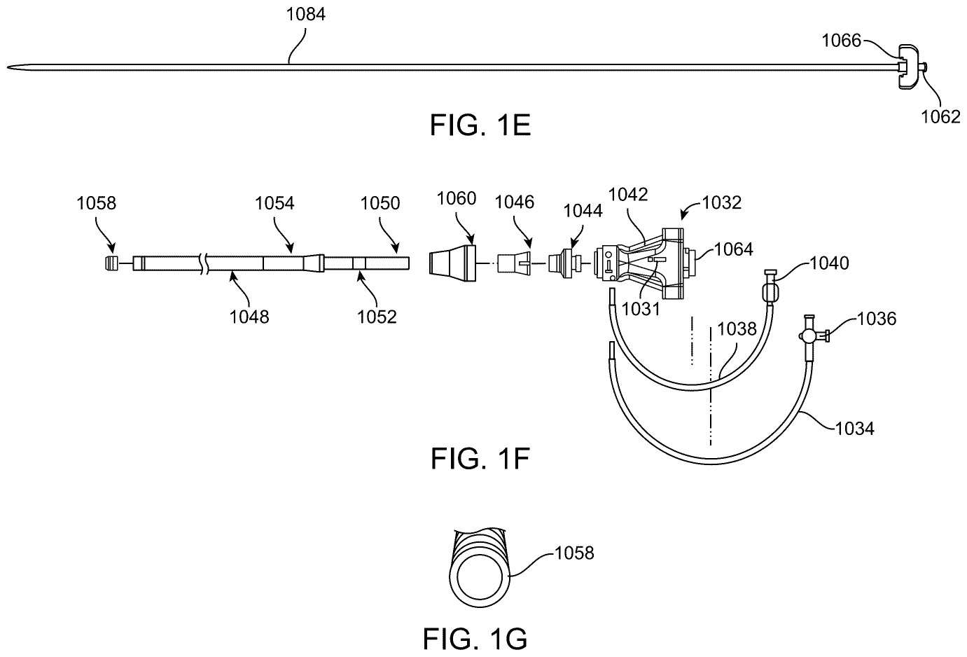

In some examples the method and apparatuses described herein may include a vacuum line with one or more of: an optional hand-triggered vacuum activation valve (in some cases the valve may be a controller for the pump on/off) and/or an optional clot capture chamber for visualizing and removing clot material aspirated by the system. FIG. 1A schematically illustrates one example of an apparatus (e.g., system), which in this example, includes a rigidizing aspiration sheath catheter 102 that includes a proximal region configured as a hemostasis valve region 106. The rigidizing aspiration sheath catheter 102 may be configured to be changed between a flexible state (flexible configuration) and a less flexible, e.g., rigid, state (rigid configuration). Any appropriate structure for rigidizing may be used, including in particular a layered structure that is rigidized by the application of positive and/or negative pressure. For example, these apparatuses may be configured as a rigidizing aspiration sheath catheter 102 configured to couple to a source of positive and/or negative pressure 112, e.g., through a port or inlet on the proximal end (which may be part of the hemostasis valve region or separate from it) to control the rigidity of the rigidizing aspiration sheath catheter 102. In some examples the rigidizing aspiration sheath catheter apparatus 102 has an elongate body comprising lumen extending therethrough. The elongate body may include layers, such as a rigidizing layer and a bladder layer that are configured to transition the elongate body between a flexible state and a rigid state by the application of pressure. As described in greater detail below, the bladder layer (e.g., “bladder”) may be driven against (or allowed to move away from) the rigidizing layer to control the flexibility/stiffness of the elongate body. The rigidizing layer may comprise a plurality of overlapping filament lengths that are free to slide over each other in the more flexible state(s), but may the bladder layer may be driven against the rigidizing layer, and/or against a support layer (e.g., a reinforced layer) on an inner or an outer region of the elongate body, to rigidize the elongate body. The pump (e.g., peristaltic pump assembly) is not shown, but examples of which are provided below.

The rigidizing aspiration sheath catheter 102 may be used with one or more obturators 132. In FIG. 1A the obturator may be inserted into the rigidizing aspiration sheath catheter 102 over a guidewire 105 (which may be included with the apparatus 1010 or may be separately provided). This may allow the rigidizing aspiration sheath catheter 102 to be guided over a guidewire positioned in a body vessel. The obturator may be steerable or not. In general, the obturator may be flexible so that the combined obturator and rigidizing aspiration sheath catheter 102 (in the flexible configuration) may readily track over a guidewire. The obturator may be longer than the rigidizing aspiration sheath catheter 102 (e.g., by more than 1 cm (e.g., between 1 cm-20 cm, between 1 cm-30 cm, 1 cm-40 cm, etc. or any number therebetween) to allow tracking while avoiding “fishmouthing” (e.g., ovalization of the tip of the outer catheter when tracking over an inner catheter/body) over the rigidizing aspiration sheath catheter 102 distal end opening. The obturator may have an atraumatic tip. The obturator may have regions of different material properties (e.g., stiffnesses), such as described in PCTUS2022082141, filed Dec. 21, 2022, and herein incorporated by reference in its entirety.

The apparatus 1010 (e.g., system) in FIG. 1A may also include one or more aspiration catheters 104 that may also include a proximal hemostasis valve region. The aspiration catheter 104 may also be used with an obturator 134 that may be inserted through the aspiration catheter 104 and inserted through the rigidizing aspiration sheath catheter 102, e.g., in the rigid configuration. In some cases it may be beneficial for the distal tip region of the aspiration catheter 104 to be directional (e.g., bent, curved, etc.) in a fairly rigid bend, to allow for directional aspiration when extended from the rigid rigidizing aspiration sheath catheter 102. The aspiration catheter 104 may generally be configured to have a relatively high flexibility with a high torquability. The high torquability may allow the apparatus to be steered (directed) within the rigidizing aspiration sheath catheter 102 when extended distally of the distal end of the rigidizing aspiration sheath catheter 102, e.g., in the rigid configuration.

Other system components may include tubing (suction line) connecting the rigidizing aspiration sheath catheter 102 and/or aspiration catheter 104 to a source of aspiration 124. The rigidizing aspiration sheath catheter 102 and/or aspiration catheter 104 may be connected via a sealing connection to the suction line, which may be connected in-line to a clot collection chamber 120, and/or a suction (e.g., vacuum) activation valve, which may be activated to apply the suction to the rigidizing aspiration sheath catheter 102 and/or aspiration catheter 104. The apparatus may also include a blood collection chamber 122 before the source of aspiration 123 (e.g., suction pump). These components may be arranged between the rigidizing aspiration sheath catheter 102 and/or aspiration catheter 104 and the source of aspiration in any appropriate order.

For example, FIG. 1B illustrates an example of an apparatus (e.g., a system) for clot aspiration including these components. In FIG. 1B the system 1010′ includes a rigidizing aspiration sheath catheter 1002 that is shown coupled to an insufflator 1012 to control transitioning between a rigid state and a flexible state. The aspiration system 1010′ also include an aspiration catheter 1004. The dynamically rigidizing aspiration sheath catheter 1002 includes a hemostatic valve region 1006. The hemostatic valve region includes a connection 1008 to a pressure source (e.g., insufflator 1012). The aspiration catheter 1004 extends proximally from the hemostatic valve region to an aspiration catheter handle 1014. Through the aspiration catheter handle, the aspiration catheter comprises a connection to a tube or other elongate element 1016 that connects to an optional vacuum activation valve 1018. In some cases the vacuum activation valve is a controller that does not occlude the aspiration line 1016, but instead turns on/off the aspiration pump 1024, which may be any of the peristaltic pump assemblies described herein (e.g., a vacuum augmented peristaltic pump). The tube 1016 extends proximally to a clot capture chamber 1020. As mentioned, the vacuum pump 1024 (e.g., a peristaltic pump assembly) may be positioned along or in some cases at a proximal portion of the aspiration lumen 1016 and blood collection container 1022.

FIG. 1C shows another example of a portion of a system 1010″ as described herein, including an aspiration catheter 1002 with an integrated hemostatic valve 1026 and a flush port 1036. The aspiration catheter is shown locking coupled to a mating attachment 1016 at a distal end of vacuum line. The mating attachment is configured to couple to a mating attachment connector on the distal end of the aspiration catheter for making a quick connection to the suction line 1017. A hand-triggered vacuum activation valve 1018 is shown connected in-line with the vacuum line and may be easily used to turn on/off suction through the apparatus. The vacuum line is also connected to a clot capture chamber 1020, described in greater detail below.

FIGS. 1D-1G show embodiments of components of an aspiration system (e.g., like those shown in FIG. 1B). Referring now to FIGS. 1D and 1F, the system may include a rigidizing aspiration sheath catheter 1030 including a hemostatic seal region 1031 at a proximal end. The hemostatic seal region 1032 comprises a body 1031. The seal in this example also includes a tube or elongate element 1034 connecting to a flush port 1036. In some embodiments, the flush port can comprise a luer type adapter. The hemostatic seal region 1032 also includes a tube or elongate element 1038 with a connector 1040 at its proximal end for connection to a pressure source (e.g., an insufflator). The connector may be a luer type connector.

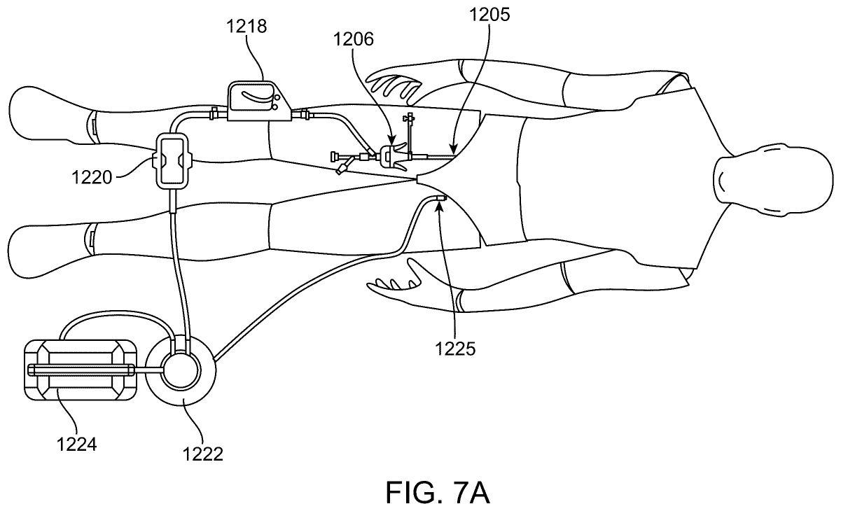

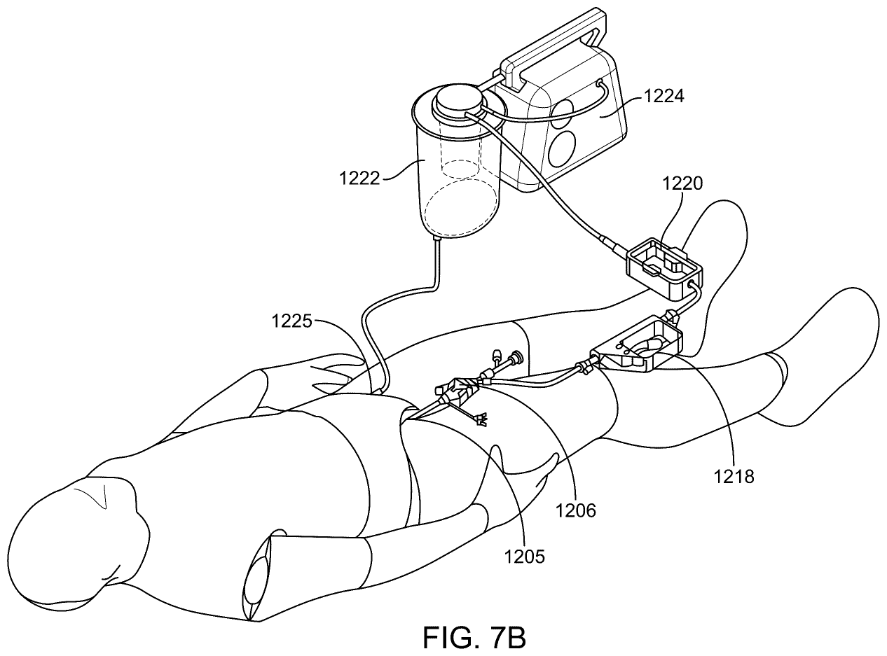

The hemostatic seal region in this example includes a pair of actuators 1042 (shown as levers). Depressing levers 1042 can allow for release of a device (e.g., aspiration catheter) positioned within the rigidizing catheter 1030. When the levers are in their unbiased state, extending outwardly from the body of the seal 1032, the seal valve is closed (shown in FIG. 7D, below).

In FIG. 1F a bladder adapter 1044 for connecting the bladder of the rigidizing aspiration sheath catheter 1030 to the seal 1032 is located a distal end of the seal 1032. This connection allows the seal 1032 to maintain pressurization (e.g., insufflation) of the rigidizing aspiration sheath catheter 1030. Distal to the bladder adapter is an adapter 1046 for connection to an outer layer 1048 of the rigidizing aspiration sheath catheter 1030. A shroud 1060 is located distal to the outer layer adapter 1046, for covering the adapters 1044, 1046 and from which the rigidizing catheter 1030 distally extends.

FIG. 1F further shows an example of the layers of the rigidizing aspiration sheath catheter 1030, including the inner layer 1050, the bladder layer 1052, the rigidizing layer 1054 (e.g., in some examples the rigidizing layer comprises a plurality of filaments that cross over each other, such as, but not limited to, a braid layer, knit layer, woven layer, etc.), and the outer layer 1048. At the distal end of the rigidizing aspiration sheath catheter 1030 is a distal tip 1058.

FIG. 1G shows an end view of the rigidizing catheter shown in FIGS. 1D-1F, in which the rigidizing aspiration sheath catheter includes an atraumatic, distal tip.

In some embodiments, the rigidizing aspiration sheath catheter inner lumen comprises a hydrophilic coating. This coating can help facilitate insertion of an obturator and other devices and accessories.

In some embodiments, an outer surface of the rigidizing aspiration sheath catheter comprises a hydrophobic coating. This type of coating can help facilitate smooth motion through an introducer sheath.

The rigidizing aspiration sheath catheter 1030 may have an inner lumen diameter of the lumen of about 0.03-0.6 in. In some embodiments, the rigidizing aspiration sheath catheter 1030 inner lumen diameter is about 0.16 in. This inner diameter allows compatibility with a 12 F catheter. In some embodiments, the inner lumen diameter is about 0.2-0.3 in. This inner diameter allows compatibility with a 20 F catheter. In some embodiments, the rigidizing catheter inner lumen is about 0.34 in. This inner diameter allows compatibility with a 26 F catheter.

In some embodiments, the rigidizing catheter outer diameter is about 0.2-0.4 in. In some embodiments, the outer diameter is about 0.23 in. or about 18 F. In some embodiments, the outer diameter is about 0.34 in. or about 26 F. In some embodiments, the rigidizing catheter as a length of about 70-120 cm (or about 80-115 cm, or about 85-115 cm, etc.). In some embodiments, the rigidizing catheter has a minimum bend radius of about 1-2 in. (or about 1.5 in.)

The system can include an obturator 1084 which can be used during navigation of the rigidizing aspiration sheath catheter 1030. Examples of obturators 1084 are described in International Application No. PCT/US2023/062206, filed Feb. 6, 2023, the entirety of which is incorporated by reference herein. The obturator 1084 can comprise a connector 1062 at its proximal end. The obturator 1084 may be configured to be inserted into the rigidizing aspiration sheath catheter 1030 through the hemostatic seal. Once the obturator 1084 is completely inserted within the rigidizing aspiration sheath catheter 1030, the obturator 1084 can be rotated to lock it in place with respect to the hemostatic seal. The rotation of connector 1062 relative to a threaded connection 1064 on the hemostatic seal region may create a lock between the mating mechanism 1064 and corresponding mating mechanism 1066 (e.g., thread, bayonet connection, etc.) on the obturator connector 1062. In some embodiments, the connector 1062 and obturator 1084 are rotated about 90° relative to one another. Other amounts of relative rotation (e.g., about 30-360° are also contemplated).

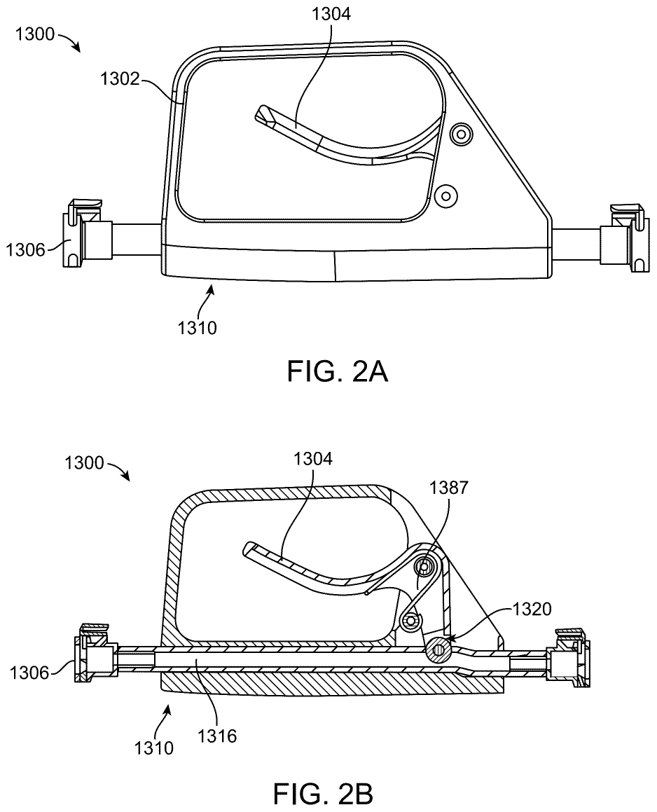

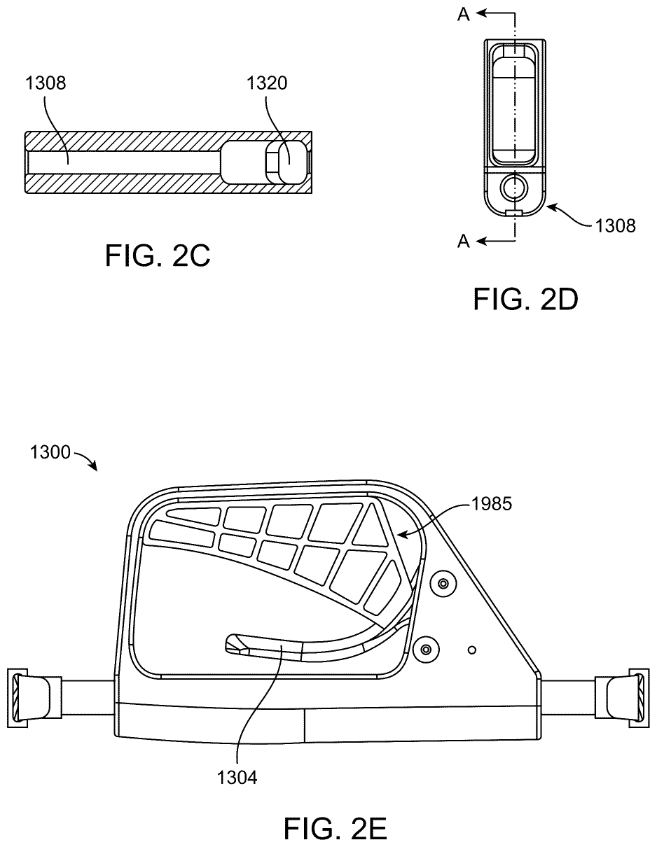

Also described herein are optional hand-triggered vacuum activation valves. FIGS. 2A-2E illustrate example views of an embodiment of a vacuum activation valve 1300. The hand-triggered vacuum activation valve 1300 includes a handle region 1302 and actuating lever 1304. FIG. 2A shows a side view of the vacuum activation valve 1300. FIG. 2B shows a side sectional view. FIG. 2C shows a sectional view taken through the base region of the handle shown in FIGS. 2A and 2B. A front view is shown in FIG. 2D.

The hand-triggered valve 1300 includes a lower portion 1310 through which the vacuum line 1316 extends. A groove or channel 1308 configured to receive that vacuum line is shown in the section view of FIG. 2C. A clamp 1320 is positioned over the channel 1316. The clamp 1310 is configured to clamp down on the vacuum line when the lever 1304 is actuated. In some examples, the clamp 1320 is configured to unclamp the vacuum line when the lever 1304 is actuated. In the un-actuated state the clamp may be configured to pinch down on (and close off) the vacuum line.

Positioned above the lower portion is a handle 1302 comprising a loop extending distally from a proximal portion of the handle. The loop can be sized to be able to be gripped within the palm of a user. The lever 1304 extends proximally from a proximal portion of the hand-operated valve 1300. The lever 1304 is sized such that it can be actuated by one or more fingers of a user while the handle 1302 is being gripped within the palm of the user. A distance between the handle 1302 and the 1304 is selected to enable the lever 1304 to be actuated when the user is gripping the handle 1302. In some embodiments, the distance is about 0.3-1.5 inches. The loop (as shown in the side view) may comprise a number of shapes (polygonal, rectangular, ovular, etc.).

In FIGS. 2A-2D the lever 1304 is shown in an unactuated state, so that the clamp pinches the channel 1316 shut. In some examples the lever and/or the clamp may be biased in the closed state by a bias 1387 (shown as a spring bias). When the lever is actuated, e.g., by pushing it down, the clamp 1320 may be pulled open, away from the channel 1316, allowing suction to pass.

In some cases it may be helpful to store a vacuum activation valve 1300 with the control (e.g., lever 1304) in a closed/actuated position. This may prevent deformation of the valve (e.g., clamp, crip, etc.) mechanism and/or the channel 1316. For example, FIG. 2E shows an example of a vacuum activation valve 1300 in which a retainer 1395 is coupled thereto, to hold the lever 1304 in a closed position, maintaining the channel within the vacuum activation valve in an open position. The retainer may be removed before use, e.g., once connected to the fluid line.

The valve can have any appropriate length. In some examples the valve has a length of about 4-6 in (or about 3-5, 5-7, 5-6 in., etc.). The handle may be any appropriate length. For example, the length of the handle can be about 3-5 in. (or about 2-4 in., 4-6 in. 4-5 in., etc.). The length of the lever can be, e.g., about 3-5 in. (or about 2-4 in., 4-6 in. 4-5 in., etc.). The lever can be curved as shown in FIG. 2A. In other embodiments, it is straight or angular.

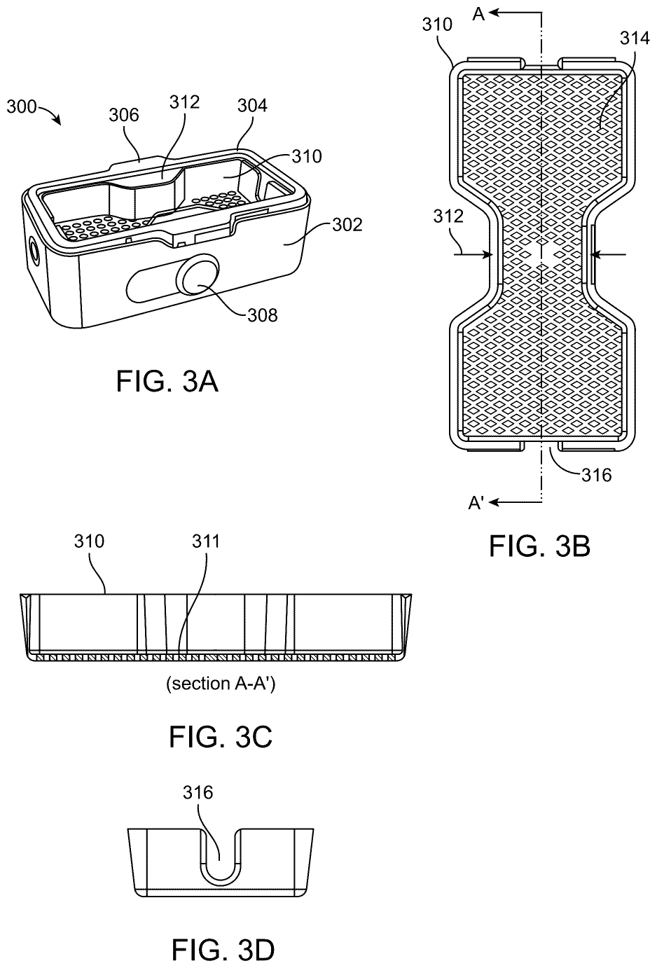

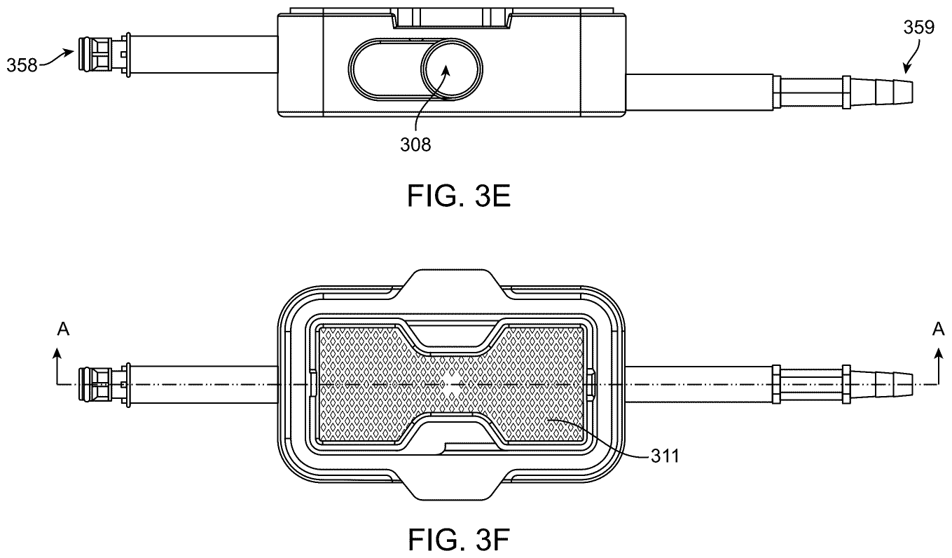

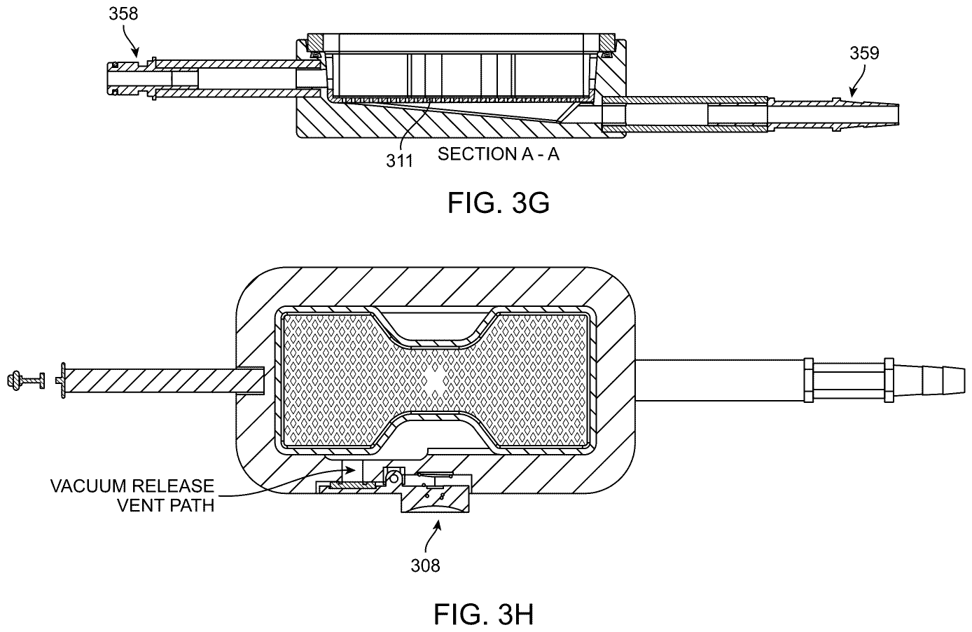

Also described herein are clot capture chambers (also referred to as clot capture containers). The clot capture chambers may be configured to capture and clot material from the patient's blood to allow for viewing or physician access. FIGS. 3A-3D show views of an example of an embodiment of a clot capture chamber (e.g., like that shown in FIG. 1B). FIG. 3A is a perspective view of a clot capture chamber. The clot capture component 300 comprises a container 902 with a lid 904. The lid 304 can be completely removed from the container 302. In some embodiments, the lid is connected to the container by a hinge. The lid 304 can comprise one or more tabs 306 extending or projecting from an edge of the lid to aid in lifting and/or removal of the lid. In some embodiments, the lid is transparent, which can advantageously allow for visualization of aspirated material therethrough.

When under negative pressure from an attached vacuum source, the lid may be sealed to the container. To enable removal of the lid, a user may perform a vent to atmosphere to release the negative of pressure within the container. In some embodiments, this venting is controlled by a button 308 positioned on the container. The container 302 can have a generally rectangular prism shape. Other shapes are also possible (e.g., square prism, elliptic cylinder, etc.). In some embodiments, the container 304 comprises a length of about 3-5 in. (or about 2.5-5.5 in., 3.5-4.5 in., about 4 in., etc.). The container can have a width of about 1-3 in. (or about 0.75-3.25 in., 1.5-2.5 in., about 2 in., etc.). The container can have a depth of about 0.3-1.1 in. (or about-4-1.0 in., 0.5-0.9 in., 0.6-0.7 in., etc.).

Positioned within the container is a tray 310. FIG. 3B shows a top view of the tray 310. FIG. 3C shows a section view taking along line A-A of FIG. 3B. FIG. 3D shows a front view of the tray 310. In some embodiments, the tray 310 is removable from the container 302. The tray 310 is sized to conform to an inner perimeter of the container 302. The tray 310 may have one or more regions 312 where it is spaced away from an inner perimeter of the container 302 to enable easy removal of the tray 310 from the container.

The tray 310 includes one or more intermediate surface(s) 311 having apertures 314 to allow for draining of blood and other fluids. In some embodiments, the intermediate surface comprises a screen or mesh like configuration. The apertures may be any sized to permit fluid (e.g., blood) to flow through, but retain clot material. The apertures (e.g., mesh) may be any appropriate size. For example, the apertures may be between about 0.037 mm and about 6 mm (e.g., between about 0.05 mm and about 5 mm, between about 0.1 mm and about 4 mm, between about 0.2 mm and 3 mm, between about 0.3 mm and 2.5 mm, between about 0.5 mm and about 4 mm, between about 0.75 mm and about 4 mm, etc.). In some examples the intermediate surface may include multiple layers of mesh having different mesh sizes in order to progressively filter out smaller and smaller clot sizes without clogging the chamber. The different layers may be separated by a gap (e.g., of between about 0.5 mm to 2 cm). These mesh regions may be removable to allow analysis and/or capture of the different size clots and/or cleaning (including sterilization).

The tray 310 may include channels 316 along two opposing sides to allow access to a vacuum lumen. The channels 316 may be U-shaped. This shape may allow the channels to partially surround a vacuum aperture on a side of the container.

The tray can have a length of about 3-5 in. (or about 2.5-5.5 in., 3.5-4.5 in., about 4 in., etc.). The tray can have a width of about 1-3 in. (or about 0.75-3.25 in., 1.5-2.5 in., about 2 in., etc.). The tray can have a depth of about 0.3-1.1 in. (or about-. 4-1.0 in., 0.5-0.9 in., 0.6-0.7 in., etc.).

In some embodiments, the tray comprises a polymer, such as acrylonitrile butadiene styrene (ABS).

FIGS. 3E-3H show side, top, side section and top section views, respectively of an example of a clot capture chamber as described herein. In FIG. 3E a vacuum release valve 308 is positioned on the side of the chamber the device may be connected in-line as part of the vacuum line, so that fluid is drawn in from the top inlet line 358, so that the fluid (e.g., blood with clot material) enters the upper region of the chamber and into the tray; the blood may then drain to the bottom of the chamber and out of the outlet 359, as shown in the sectional view of FIG. 3G. In any of these clot capture chambers the inlet may be positioned at a top region, and the outlet may be positioned at the bottom region, allowing gravity to assist in passing the blood through the porous intermediate layer(s). Any of these apparatuses may include a seal (e.g., gasket) between the main body and the lid, as shown in section view of FIG. 3G.

An exemplary method of using the aspiration system described herein comprises navigating through the vasculature to or near a desired clot treatment site using a guidewire (e.g., 0.014″ or a 0.035″ wire). A rigidizing catheter can then be advanced, in a flexible state, over the guidewire to or near the clot treatment site. Once at or near the site, the rigidizing catheter may be transitioned to the rigid state. An obturator can be used during advancement of the rigidizing catheter. At this point, the guidewire may be removed, if so desired. Removing the guidewire can advantageously free up space within the lumen of the rigidizing catheter.

Once the rigidizing catheter is in the rigid state, it creates a stable conduit or platform from which treatment can be initiated. An aspiration catheter can be advanced through the rigidizing catheter. The aspiration catheter can have a proximal connector configured to lock to the hemostasis valve of the rigidizing catheter. The aspiration catheter can have a bigger inner lumen than traditional aspiration catheters because the stable platform created by the rigidizing catheter reduces the need for the aspiration catheter to perform accurate navigation and allows it to have a larger central bore. As mentioned here, the rigidizing catheters described herein may provide sufficient support (e.g., mechanical integrity) to one or more additional devices or components to allow these additional components (guidewires, aspiration catheter, stent, heart valve, tools, etc.) to be more flexible than other components. The stiffness necessary for the control and operation of a standalone component may become redundant when used within the rigidizing catheters described herein, which may provide the mechanical support that is otherwise required in the standalone component.

In some embodiments, the aspiration catheter can be switched out for a different aspiration catheter (e.g., comprising a different size). The hemostasis valve can be actuated to release the first aspiration catheter and insert the second aspiration, allowing for a quick exchange of the aspiration catheters. The aspiration catheter handle can be actuated to release the first aspiration catheter from the vacuum line and a second aspiration catheter can be connected to the vacuum line.

In some embodiments, the aspiration catheter may be removed to allow for flushing of the aspiration line. The rigidization catheter maintaining the stable pathway to the treatment site allows for this flushing to occur without losing the catheter position.

In some embodiments, the method further comprises visualizing aspirated material through the transparent lid of the clot capture container. In some embodiments, the container may be vented, and the lid removed. The tray may be replaced with another clean tray, for example, if the container is getting full. Traditional aspiration systems do not allow for a large enough aspiration lumen size to aspirate enough material to require this replaceable tray feature. A single tray can be used throughout the procedure, or multiple trays can be used—for example, one tray for each suction attempt or for each clot removed.

In some embodiments, aspiration can be performed directly through the rigidizing catheter.

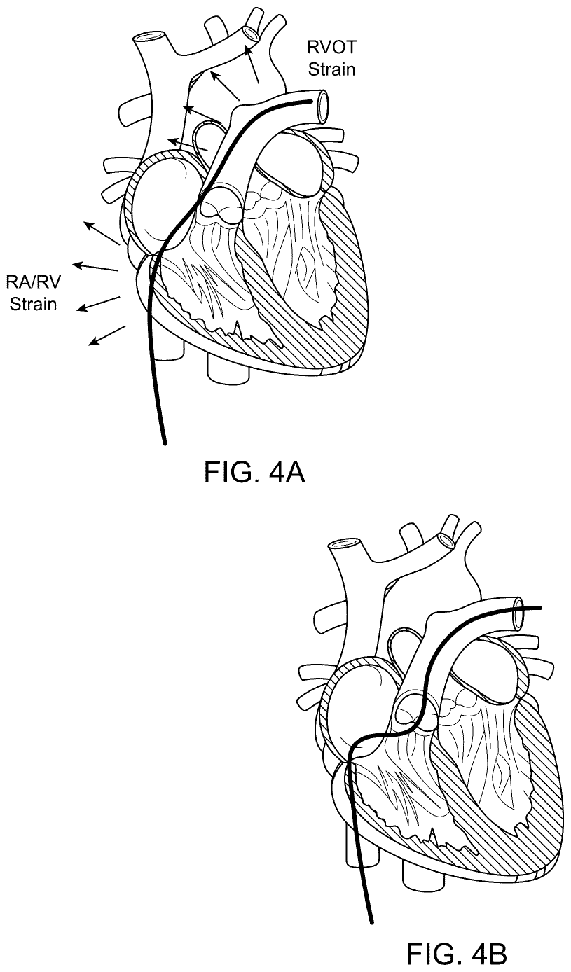

FIGS. 4A and 4B show a traditional catheter navigated to the pulmonary artery (FIG. 4A) and the rigidizing system described herein navigated to the pulmonary artery (FIG. 4B). As shown in FIGS. 4A and 4B, the rigidizing system's ability to establish the pathway in a flexible state leads to reduced strain and reduced potential for hemodynamic compromise and injury.

In general, the removal of clot material may result in the loss of large blood volumes. Blood loss can be associated with the duration of the aspiration and the proximity of the aspiration catheter tip to the clot. However, the methods and apparatuses described herein may be configured to minimize blood loss. For example, the use of rigidizing apparatuses as described herein may allow apparatuses to be positioned proximate to the clot, which may minimize the blood volume required to move the clot. The stabilization achieved with the rigidizing catheters described herein may improve access to the clot and lead to better engagement between the tip of the aspiration catheter and the clot, resulting in lower blood loss by virtue of early and improved clot engagement. In addition, the methods and apparatuses described herein may be used without the use of guidewires. In particular, the apparatuses described herein may be configured to accommodate a cardiotomy reservoir (e.g., a suction canister/blood filter), which may be integrated within a thrombectomy circuit as described herein.

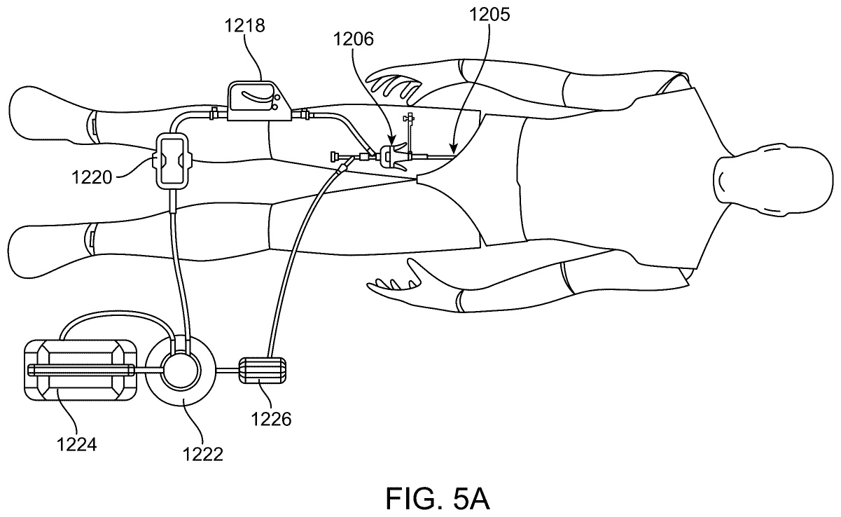

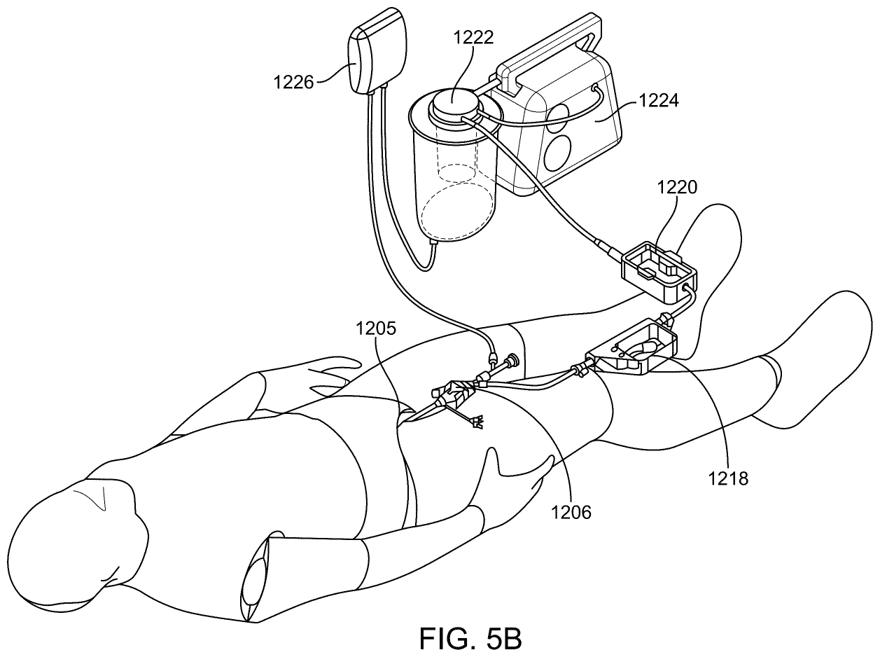

FIGS. 5A-5B, 6A-6B, 7A-7B, 8A-8B and 9A-9B illustrate examples of systems including a rigidizing aspiration sheath catheter configured with a blood return circuit. In general, these apparatuses may be configured for use with a patient to remove, aspirate material, and in particular clot material, from the vascular system of a patient. In any of these examples the apparatus may include a return circuit for returning and/or replacing blood removed during the procedure. For example, FIG. 5A illustrate a system as described herein including any of the rigidizing aspiration catheters 1206 described herein. The aspiration catheter may be coupled to a hand-operated valve (extraction handle 1218) that connects the aspiration tube in-line with the clot capture container 1220, which is in turn connected in-line with a suction canister including one or more blood filters 1222. The suction cannister may be held under vacuum to a negative pressure set by a vacuum pump 1224.

The suction cannister may filter and/or treat the blood so that it may be reintroduced into the body. For example the suction canister 1222 may include filtration to remove clot material and/or may be treated with one or more agents to reduce or prevent infection and/or to reduce and prevent clotting (e.g., anticoagulants). As mentioned above, the optional clot capture 1220 device may also filter clot material before it reaches the suction canister 1222. In general the filtered and/or treated blood may be reintroduced back into the body either directly or indirectly, e.g., by first passing to a blood bag 1226. Supplemental blood may be provided.

In general, the sterility of the operating sterile field may be maintained by keeping all of these elements (e.g., the blood bag, suction cannister, clot capture device, handle, etc.) within the sterile field. Any or all of these components may be single-use and/or reusable (and sterilizable).

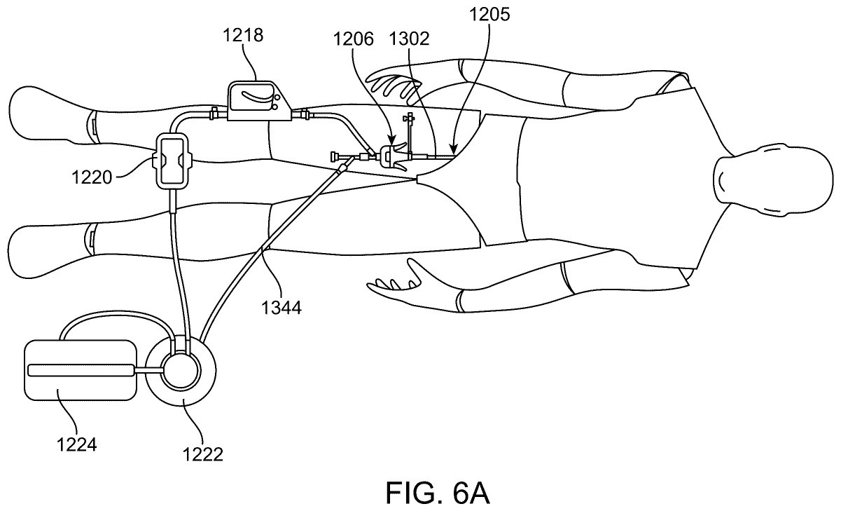

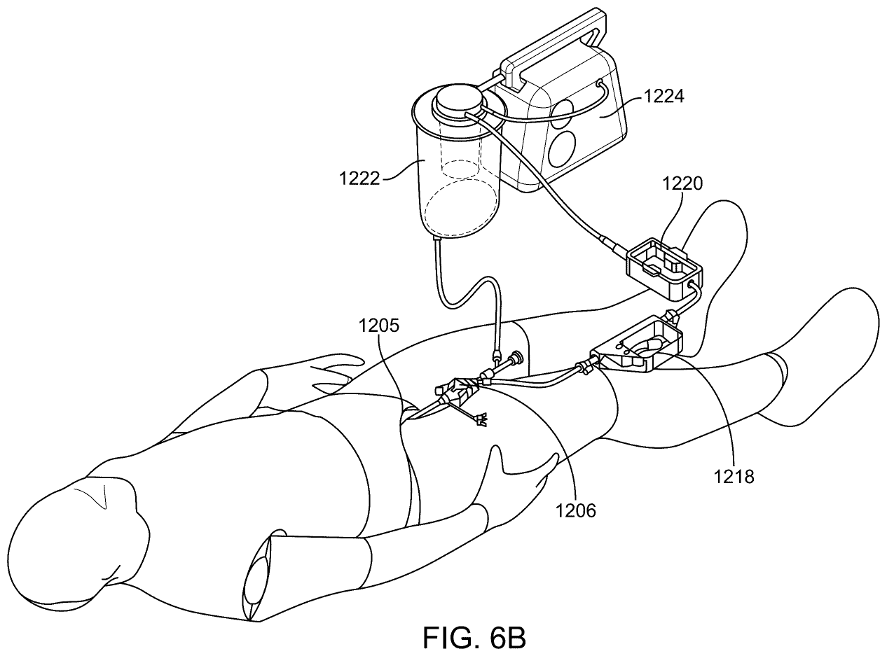

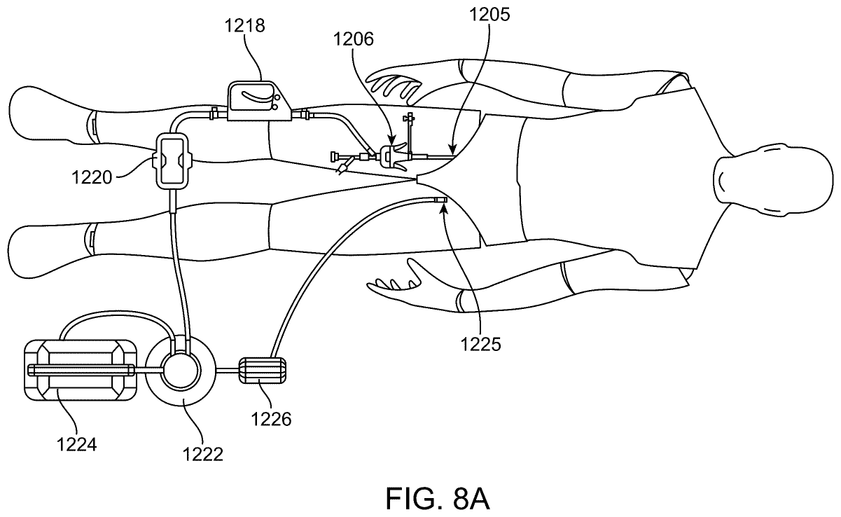

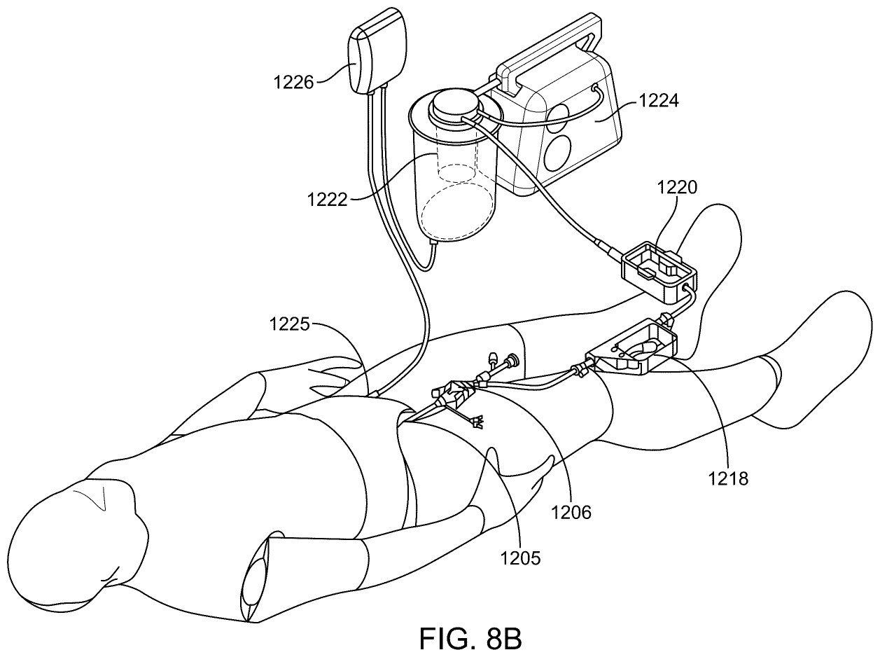

The rigidizing aspiration catheter may be inserted into an access site 1205 on the patient's body, such as the femoral artery. Note that any appropriate access region may be used (e.g., radial, ulnar, axillary, brachial, dorsalis pedis, posterior tibial). In FIGS. 5A-5B and 6A-6B the system is configured so that the material, including blood, is removed and returned via the same access site 1205. Alternatively, in some examples the return site may be a separate access site, such as illustrated in FIGS. 7A-7B and 8A-8B. In these examples the blood return site 1225 may be on the contralateral side (e.g., the contralateral femoral artery).

For example, FIGS. 6A-6B illustrate a configuration of an apparatus including a rigidizing aspiration sheath catheter 1302, which includes a hemostasis valve at the proximal end coupled to a suction connector 1206. The suction connector fluidically connects to the control valve (e.g., configured as an actuation handle 1218), in-line with a clot capture chamber 1220 and a blood capture/filter chamber 1222 that is connected to a source of vacuum 1224, similar to FIGS. 5A-5B. In this example, the rigidizing aspiration sheath catheter may be inserted through the access site 1205 and navigated through the body (e.g., over a guidewire) including as shown in FIGS. 4A-4B, across the heart, to a target region to remove clot. Once in position, the rigidizing aspiration sheath catheter may be rigidized, e.g., by applying positive and/or negative pressure, and coupled to the suction line using the connector 1206. Aspiration may be applied, e.g., by actuating the control valve 1218, with the device maintained in the rigid configuration (e.g., by maintaining the positive and/or negative pressure).

In some examples, after aspirating one or more times, by activating and releasing the extraction handle 1218, a state of vacuum may be re-established between extraction handle and the blood capture chamber 1222 (e.g., reservoir), including the portion of the blood return line up to the patient; a check valve may be included on the blood return line. Blood may be held by the blood capture region. In any of these examples, an optional blood return circuit may be included, as shown. In this example, the blood return circuit includes a return tubing line 1344.

In any of these methods, the rigidizing aspiration sheath catheter may be used to support a second catheter, an aspiration catheter, which may be inserted through the rigidizing aspiration sheath catheter, as described above. The rigidizing aspiration sheath catheter may be uncoupled from the suction line, the aspiration catheter inserted through the rigidizing aspiration sheath catheter (e.g., using the posterior hemostasis valve of the rigidizing aspiration sheath catheter) and the aspiration catheter coupled to the suction line, all while the rigidizing aspiration sheath catheter remains in the rigid configuration. Suction may then be applied through the aspiration catheter, while the rigidizing aspiration sheath catheter remains rigid, to support the aspiration catheter.

When it is time to reinfuse blood to the patient the vacuum may be removed (e.g., bringing the system to atmospheric pressure), which may be done by pressing a release control (e.g., button) on the clot capture chamber 1220, which may leak air into the system in a controlled manner. Air and any remaining blood may be in the clot capture chamber 1220 may be driven to the blood capture chamber/filter 1222. In some cases the lid of the clot capture chamber may be taken off to maintain this state. At this point, a user could use a syringe at the check valve to pump/reinfuse blood to the patient, or a more automated blood return may be used.

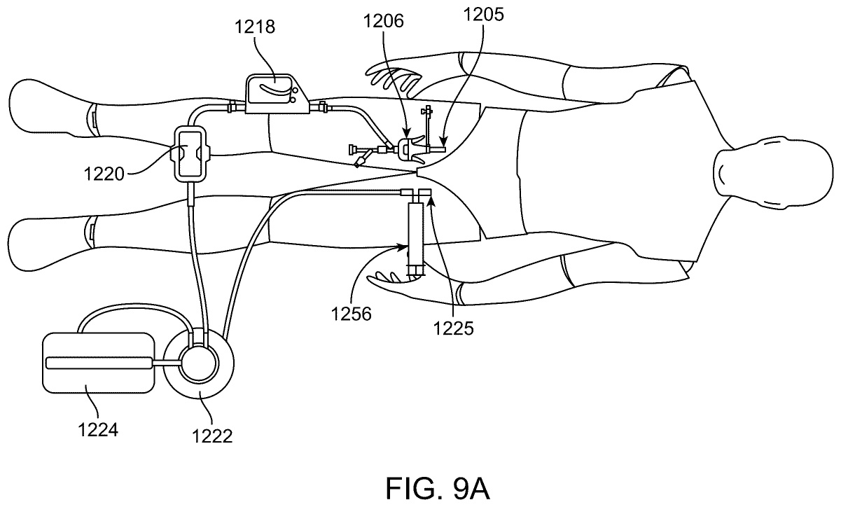

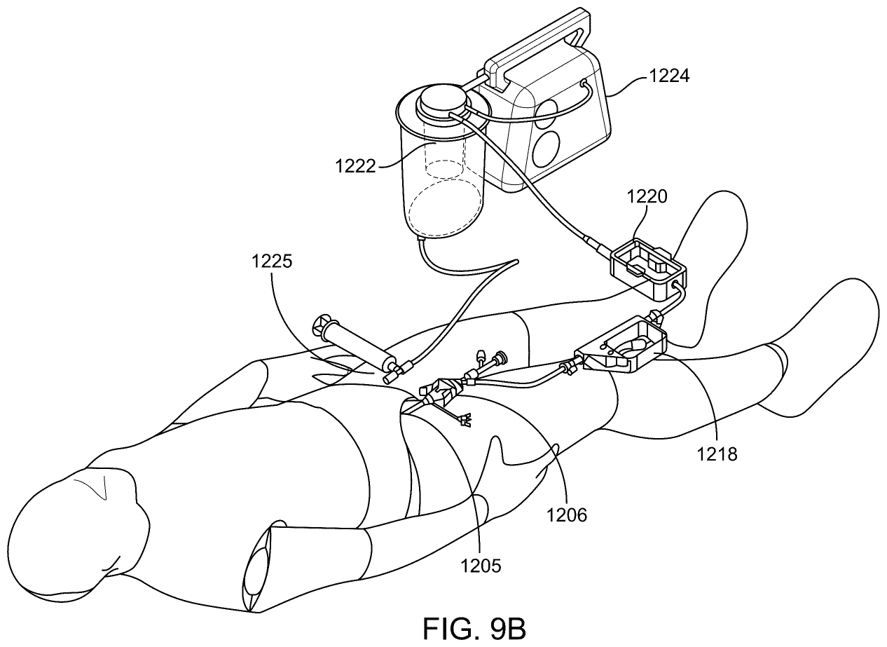

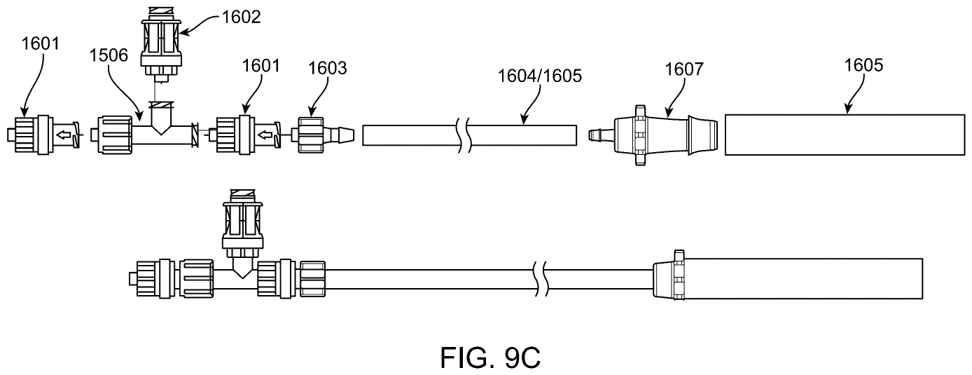

As mentioned, in some examples the blood return circuit may be direct, and may couple from the suction canister/blood filter directly back into the patient's body, as shown in FIGS. 6A-6B and 6A-6B. For example, blood may exit the patient from the catheter and may be returned to patient through either the catheter (e.g., the rigidizing aspiration sheath catheter or aspiration catheter) or alternate means (not shown). FIGS. 9A-9B illustrate an alternative site 1225 for re-introduction of blood (e.g., on the opposite leg) using a syringe 1256, as mentioned above. FIG. 9C shows an example of a return assembly 1600 including a check valve 1601, a swabble luer fitting 1602, a male luer lock 1603, tubing 1604, 1605, and a T-connector 1506 and a reducer 1607. This blood return tubing assembly may generally include multiple one-way valves arranged so that fluid may be injected into the body while minimizing blood loss.

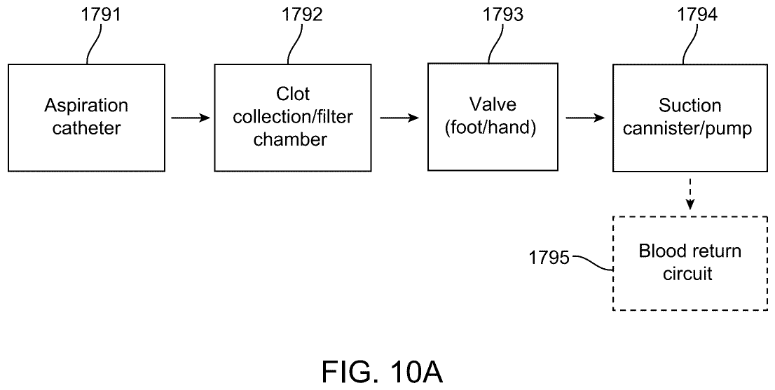

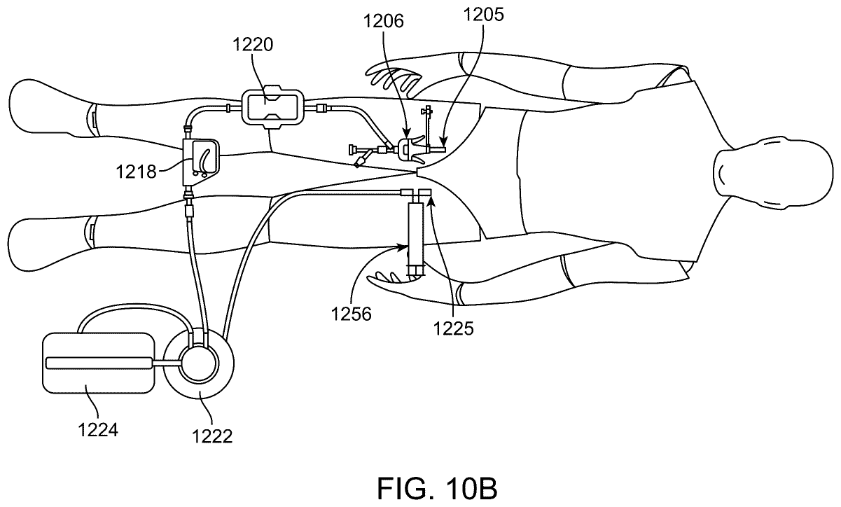

As mentioned, the order of the components of the blood circuit may be different. FIGS. 10A-10B show an example of a system in which the clot capture chamber 1220 positioned proximally to the suction valve 1218. For example, in FIG. 10A, the rigidizing aspiration sheath catheter and/or aspiration catheter 1791 may be proximal to the clot collection chamber 1792 which is proximal to the suction valve 1793 (e.g., shown as a hand valve 1218 in FIG. 10B) and the blood collection chamber (e.g., suction cannister) and pump 1794.

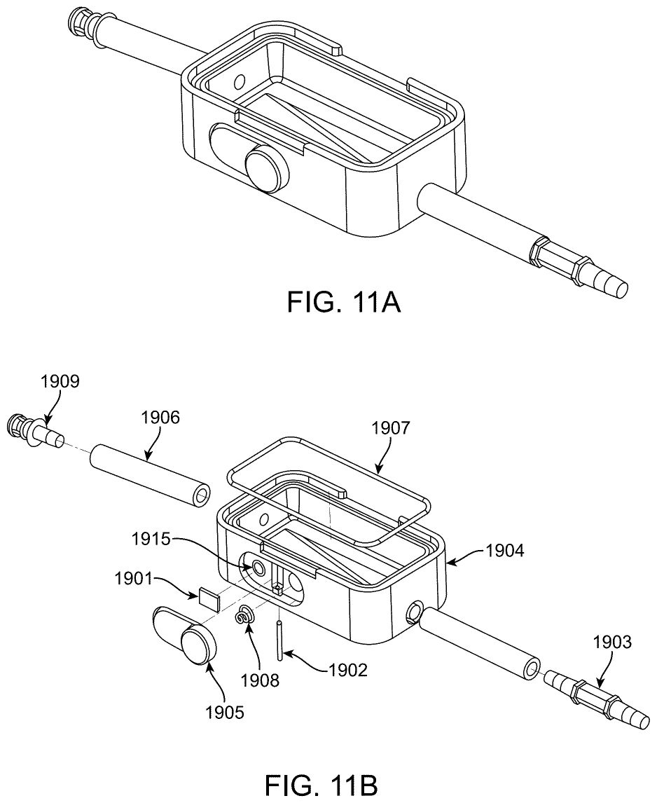

As mentioned, any of the systems described herein may include a clot capture chamber/container., The clot capture chamber may be configured to prevent or reduce hemolysis during the procedure. The clot capture chambers may be configured to infuse air into the clot capture chamber during venting at a control rate (e.g., more slowly/gradually than simply releasing/opening the system). This can be done by modulating diameter of hole beneath the venting button. FIGS. 11A-11B illustrate another example of a clot capture chamber 1900 configured to minimize hemolysis. In the example of FIGS. 11A and 11B, the clot capture chamber includes a common rotator seal 1901, a dowel pin 1902, a barb fitting 1903, a clot capture body 1904, a release (vent) button 1905, tubing 1906, o-ring 1907, a conical spring 1908, a barb fitting (male) 1909, and sealing connectors 1910, 1911. By reducing diameter of the opening 1915 controlled by the button 1905, e.g., to a range of 0.020″ to. 0.100″ slowed airflow in a way that reduces hemolysis.

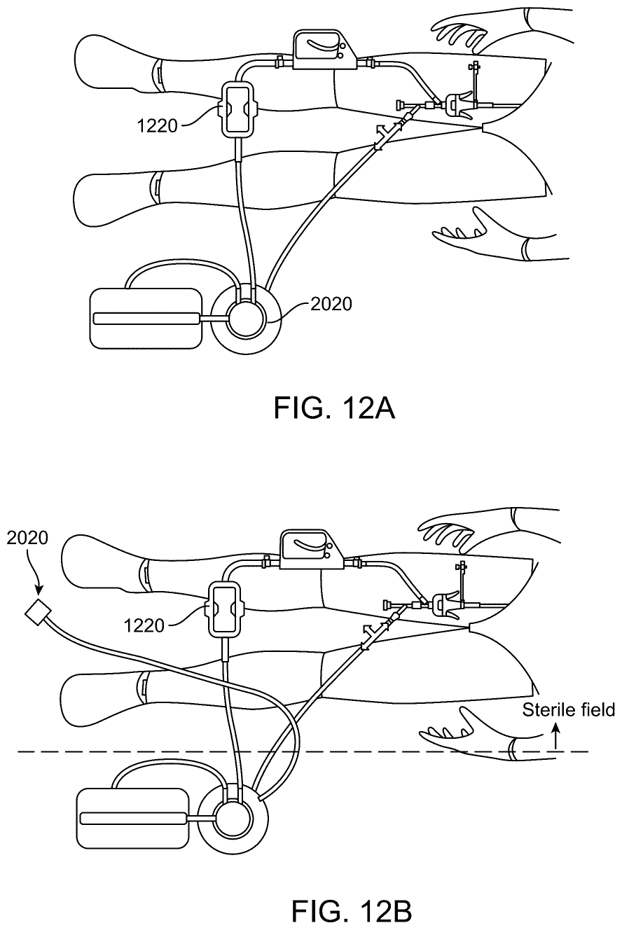

In any of these apparatuses a venting button may be positioned on top of the reservoir (e.g. the blood capture/filter chamber 1222). The venting button could be similar to the one shown in FIG. 11B in the clot capture chamber 1900, with an associated hole/orifice whose diameter is in the range of, e.g., 0.020″ to 0.100″. When it is time to eliminate vacuum from the interior this button 1905 may be pressed so that air leaks into top of blood capture/filter chamber 1222. This is illustrated in FIG. 12A. As a consequence, there would be no rushing of air and blood in the tubing. Some blood may stay behind in the clot capture device, but overall there should be a reduction in hemolysis. In some examples a tube may extend from the reservoir to the sterile field, and the vent button 2020 may be pressed on the sterile field, as illustrated in FIG. 12B. Venting valves may be momentary or latching, or have both capabilities.

### Positive Displacement Pumps

Any of the apparatuses described herein may be used with a positive displacement pump. These pumps may be particularly advantageous as they may operate on demand, without requiring a vacuum to be established before aspiration may be applied. This may reduce the likelihood of leaking and may minimize or prevent hemolysis. The use of a positive displacement pump, and in particular a peristaltic pump, may also reduce or eliminate hemolysis, which may be particularly important when filtered blood is returned to the patient.

Any appropriate type of positive displacement pump may be used, and may be specifically configured for use in removing clot, which may benefit from a relatively high flow rate and pressure. A high flow rate may be advantageous in engaging clot, while a high vacuum pressure may be helpful to pull clot into and through the catheter and proximal tubing and fittings. In general, positive displacement pumps transport fluids by mechanically trapping a fixed volume of fluid and displacing it into a discharge system. They may cyclically reduce and expand a cavity, which moves the fluid at a consistent rate, largely independent of discharge pressure. Rotary positive displacement pumps, such as peristaltic pumps, use rotational movement to trap and transport fluids, which have traditionally been thought used for applying for low-to-medium flow rates. Thus, although such pumps are used for other medical indications, these pumps have not been widely adopted of use with aspiration, an application, particularly where high flow rates (such as 1.0 L/min or more, 1.25 L/min or more, 1.5 L/min or more, 1.75 L/min or more, 2 L/min or more, 2.25 L/min or more, 2.5 L/min or more, 2.75 L/min or more, 3 L/min or more, 3.25 L/min or more, 3.5 L/min or more, 3.75 L/min or more, 4 L/min or more, 4.5 L/min or more, 5 L/min or more, 5.5 L/min or more, etc.) are believed to be beneficial. The positive displacement pumps described herein, and in particular, the peristaltic pumps, may be configured to apply such high flow rates. The flow rate may depend on catheter size, however, for a 20 F catheter, it has been found that a flow rate approaching 6 L/min may be very useful. Further, it may be particularly helpful to have both high flow and high lift (vacuum pressure) so that fluid acceleration time does not result in more blood loss or failed attempts at engagement (also resulting in unnecessary blood loss). However, contemporary high end, commercially available tubing/peristaltic pumps that are biologically compatible may use up to about a ⅜″ ID peristaltic tubing. A ⅜″ ID tube will typically max out, unless being fed by a positive pressure source, at around 4 L/min. Alternatively, ½″ ID tubing may be rated for 6 L/min or higher, but this typically has a vacuum rating that is no higher than around 25 in Hg. As mentioned, peristaltic pumps are not generally employed in clot aspiration is that when a roller engages. In addition to the limited flow and pressure profiles, typical pumps also include a brief positive pulse of pressure that can push clot away rather than draw it in, due to the pulsatile nature of the pumps. Below describes several methods of circumventing these shortcomings so that the numerous benefits of peristaltic pumps may be employed for clot aspiration. Among some, the peristaltic pumps described herein may significantly dampen or minimize the oscillation (e.g., the positive-directed portion of the pulse) by including one or more in-line elements distal and/or proximal to the pump, including the clot capture chamber, which may include air or other compressible fluid that may absorb and dampen the pulsing when applying suction.

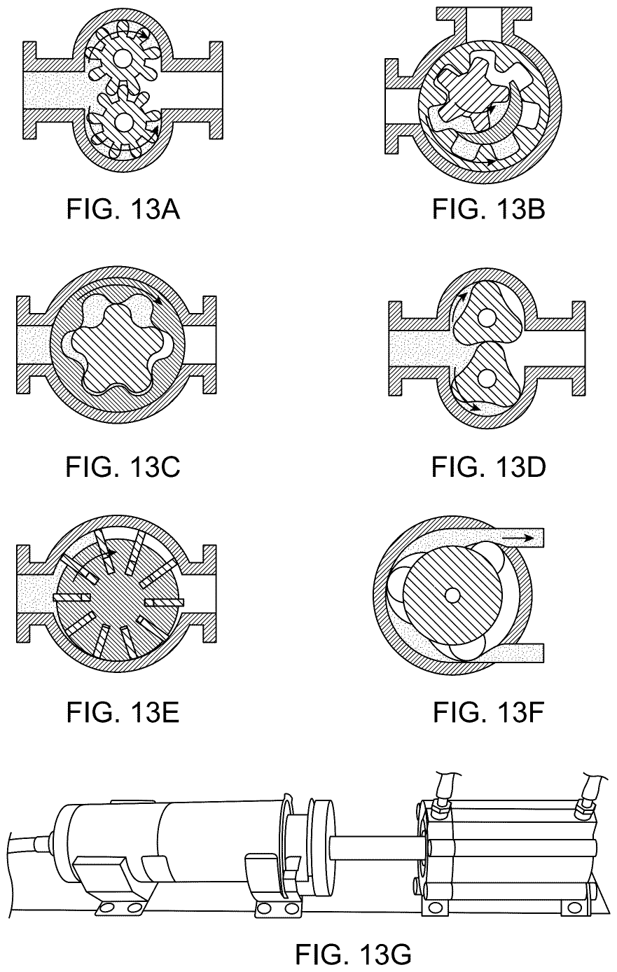

FIGS. 13A-13G illustrate examples of different types of positive displacement pumps that may be used. For example, an external gear pump (FIG. 13A) may be used; external gear pumps may use two meshing gears to pump fluid. FIG. 13B schematically illustrates another example of a gear pump, an internal gear pump, which uses an inner and outer gear arrangement. FIG. 13C shows an example of another type of internal gear pump, a gerotor pump, including an inner and outer rotor, with the inner rotor offset from the outer rotor's axis. A scroll pump and/or diaphragm pump (not shown) may also be used.

FIG. 13D shows an example of a lobe pump which uses intermeshing lobes to transport fluids. FIG. 13E illustrates an example of a vane pump that uses vanes mounted on a rotor that slide in and out to maintain contact with the pump casing. These pumps are self-priming. FIG. 13F schematically illustrates an example of a peristaltic pump, and FIG. 13G shows an example of a reciprocating positive displacement pump (e.g., a syringe pump), which includes a piston that is driven by a linear actuator. Other examples of positive displacement pumps may include screw pumps, which employ one or more helical screws, and progressive cavity pumps, which use a helical rotor inside a flexible stator.

The positive displacement pumps shown in FIGS. 13A-13G may be used with any of the apparatuses and methods described herein to provide aspiration for removing clot material (and blood), and in some cases returning blood to the patient. In variations including blood return, a separate pump (or no pump, e.g., syringe, gravity feed, etc.) may be used. In some examples the same pump may be used both for pulling (aspirating) blood from the body and for pushing (returning) into the body.

A peristaltic pump may be particularly preferred as a source of on-demand aspiration, and optionally, for blood return. It has long been believed that peristaltic pumps are unable to achieve sufficiently high flow rates to aspirate clot material, which typically benefits from very high instantaneous pressures and/or flow rates in order to dislodge and remove clot material from the vasculature, particularly the peripheral vasculature. Additionally, the pulsatile nature of traditional peristaltic flow pumps may inhibit of clot engagement, in part due to the slight positive pressure pulse generated when a roller engages the tubing. The methods and apparatuses described herein may use one or more peristaltic pump (e.g., in parallel) to provide a high flow rate and/or high pressure aspiration from one or more (e.g., parallel) peristaltic pumps. The peristaltic pumps described herein may be used without requiring exotic tubing materials that may increase the complexity, cost and reliability of these system, and may enhance the performance of existing tubing.

In general, a peristaltic pump advantageously both pulls fluid towards it as well as pushes fluid away from it. This is contrary to a vacuum pump with a reservoir which can only pull fluid (e.g., air is exhausted when evacuating a space, and care is typically taken not to put fluid through the vacuum pump, which would damage it). Pushing the fluid allows the pump to push it into a pressure vessel such as a weighted syringe, or an elastomeric bag, any vessel that can increase in volume and applies a force to decrease the volume such that blood can be forced back into the body by that vessel once a valve is opened between the vessel and the reinfusion catheter, potentially simplifying the return of filtered blood during and/or after the procedure. In some cases the pressure vessel can be a fixed volume so that adding fluid increases the pressure. In some cases the pressure vessel can be independently pressurized/maintained at a particular pressure and the pressurized output of the peristaltic pump overcomes this pressure.

Peristaltic pumps may naturally seal the input and output sides of the pump/tubing, without the use of a valve; the rollers that push and pull fluid (and/or soft solids, like clot) through the tubing pinch off discrete regions of tubing forming the blood circuit with the catheter removing the material. Once the media within the tubing reaches past the roller of the pump, the media can be pushed with a higher pressure than 1 atm (limit of vacuum) thus it can move more viscous or more solid materials. It can also break up soft things such as a clot into smaller chunks to reduce the likelihood of clogging without compromising the tubing or damaging the pump.

The peristaltic pumps described herein may move fluid through a completely closed path where the fluid within only contacts highly controlled and clean surfaces (tubing) and no moving parts such as the sliding seal of a syringe. In contrast, vacuum pumps generally cannot withstand any fluid ingress into the pumping mechanism itself and therefore must have a reservoir external to the pump to capture the fluid. The reservoir may be vented and fluid must then be pumped out of that container to further manage the fluid.

The peristaltic pumps described herein also limit mixing of the blood with air, which may be advantageous for reducing hemolysis if the blood is to be returned to the patient. Blood is only under vacuum during the extraction process; and may only see high vacuum levels when pulling on a stubborn clot. In contrast, a traditional vacuum pump system typically includes a reservoir of blood under constant vacuum which could cause hemolysis. This may reduce the likelihood of foaming of the blood that would otherwise occur in a vacuum pump and reservoir based system. Returned blood may also have a higher oxygen content since less of it spends time under vacuum. This may be beneficial for a compromised patient.

As described herein, vacuum generation can be done on demand rather than charging an evacuated container with vacuum. In addition, peristaltic pumps can ramp up pressure and flow much faster than a vacuum pump as they do not need to remove air from a chamber in order to create suction and fluid motion, permitting on-demand aspiration. Peristaltic pumps are also typically reversible, and able to move fluid one direction and then back the other direction.

The peristaltic pumps described herein may also isolate the fluid-contacting components so that they can be disposed of after a procedure, while the core pump may be reusable. In some cases, the peristaltic pumps described herein may be included within a sterile container from which the pump can be removed after a procedure and be reused for another case by inserting into a fresh (and potentially disposable) sterile container. For example, the peristaltic pumps described herein may sit in a sterile barrier, which may allow the pump to be reused but still be in the sterile field to reduce blood loss, which may also permit shorter lengths of tubing (e.g., 1.5 m or less, 1.3 m or less, 1.2 m or less, 1.1 m or less, 1.0 m or less, 0.9 m or less, 0.8 m or less, 0.7 m or less, 0.6 m or less, 0.5 m or less, etc.) between the pump and the catheter (which may in some cases include a clot capture container).

Any of the peristaltic pumps described herein can be controlled with a user actuatable switch (e.g., a hand controlled and/or foot controlled switch). When on, the pump immediately turns on to move fluid. When off, the pump is not moving fluid. Vacuum moving the blood/clot is not stored within the peristaltic pump, but is generated only when the pump is on. Any of these peristaltic pumps may include a mechanical valve between the vessel and the pump where, at the onset of valve opening, the pump immediately begins pumping. The pump can be designed to have a single pulse (e.g. on-time) per user actuation, or continuous motion or a combination thereof. The pump may be configured so that the user pushes the button and for the pump to perform one cycle that removes a specific volume of blood at a high flow rate. Alternatively or additionally, the pump may be switched on continuously until turned off, or turned on for a specific time. The pump may be configured to have an initially high flow rate upon user actuation, but then quickly reduce to a lower flow rate. For example, if the user holds the button down for 3 seconds, the first 0.5 seconds may be approximately 5 L per minute and the remaining 2.5 seconds may be at 0.5 L per minute. Pump operation may be open loop, as described above, or have some feedback triggering different actions. For instance, if after 0.5 seconds, the negative pressure does not reach at least 50% of full vacuum, the pump shuts off. If it does reach 50% of full vacuum, the pump continues at a reduced flow rate until the vacuum level drops below 50% of full vacuum. This may allow for ideal clot engagement upon initial push/pull (first 0.5 seconds) followed by high vacuum pressure but low flow for 2.5 seconds so as to not drain too much blood if the clot was not engaged. If the clot is engaged, high flow is no longer required, therefore the user can continue to hold down the button and the pump will continue to work with high vacuum pressure and low flow rate. This may be advantageous to pull the clot through the catheter (which may require high vacuum but very little flow rate). If the pump continues to operate at a high RPM, when the clot clears, the operation or system may stop the pump and prevent unnecessary blood loss.

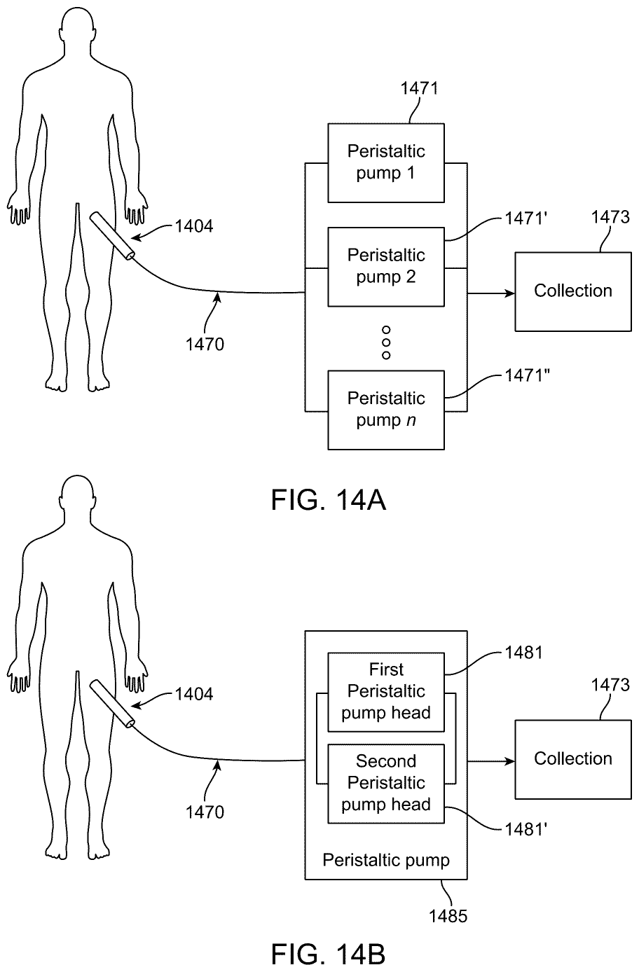

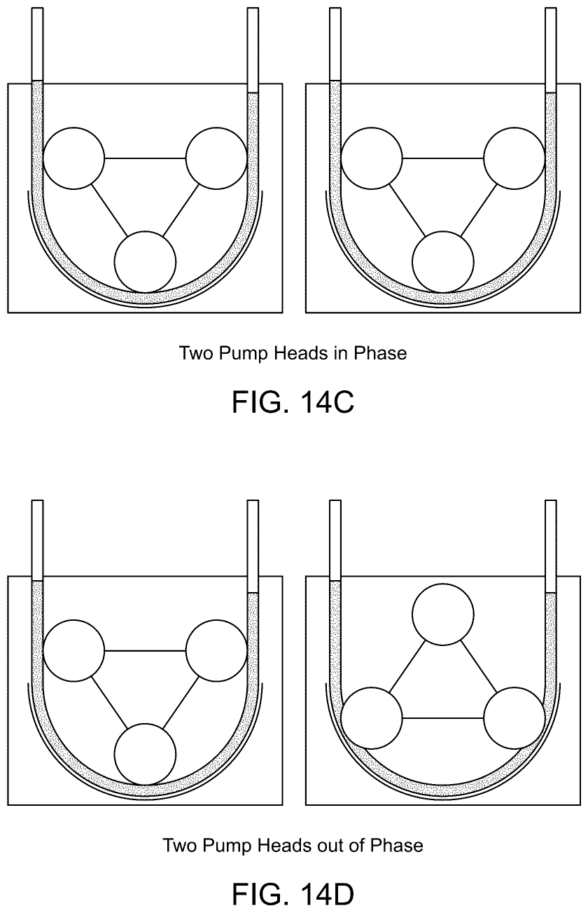

In some cases, the pump may have multiple circuits that run in parallel for increased flow rate. For example, a single motor can be connected to run 3 roller and tubing circuits at the same time. Flow may be split from one catheter to the 3 circuits then back to a single tube for collection in a reservoir. This increases the available flow rate by 3 times without negatively impacting vacuum pressure. The two or more peristaltic pumps could also be two or more independently controlled and motorized pumps (each having a separate rotor and/or motor), as shown schematically in FIG. 14A, or equivalently, two or more peristaltic pump heads that share a single rotor and/or motor, and may be part of the same housing. In this example, n individual peristaltic pumps 1471, 1471′, 1471″ (e.g., n may be 2, 3, 4, 5, 6, etc.) are arranged in parallel and connected to a single fluid line 1470 an aspiration catheter 1404. The fluid line may divide into individual lines coupled to each peristaltic pump. A clot collection chamber (not shown) may be coupled either distal to the pump(s) or proximal to the pumps and the pumps may output into a blood collection chamber 1473; the collected and filtered blood may be returned to the patient immediately or later. These pumps may be in the same housing, and may be controlled by a single controller. In some cases multiple buttons or positions of a button/user control to activate all of them or some of them depending on what flow rate the user wants. In some examples the pump heads may be synchronized/in phase (e.g., the rollers engage tube at the same time) to maximize pulsatile flow, out of phase (e.g., each rollers' relative position to the other pump's rollers are arranged such that only one roller is engaging at a time) so as to minimize pulsatile flow, or a random of roller positions. The pumps rollers may also be able to switch between synchronized/in phase and out of phase operation. This is illustrated in FIGS. 14C and 14D. FIG. 14C shows two pump heads (which may share a single motor and/or rotor or may have different motors that are coordinated) are rotated in phase. In FIG. 14D two pump heads (which may share a single motor and/or rotor or may have different motors that are coordinated) are shown out of phase. The use of out-of-phase roller pump heads may reduce and/or eliminate regurgitation/positive pressure spikes, although there may still be pulsatile oscillations in flow/pressure, which may be damped as described herein.

In any of these apparatuses the fluid lines could be fitted with pressure sensors to throttle back or stop the pump when clot clears the tubing, reducing unnecessary extraction of blood. For example, the apparatus may include a shutoff/throttle that may control the pump.

Any of these apparatuses may include a control (e.g., button, valve, etc.) that relieves vacuum and also closes off fluid communication between a clot capture chamber and the catheter or relieves positive pressure and closes off communication to the reservoir, if on the positive pressure side of a pump. As described above, the lid of the clot capture chamber can be opened to remove the clot from the clot capture. In some cases the lid may close off communication between catheter and clot capture chamber then vents clot capture chamber to atmosphere, then it unlocks the lid. If before the pump, this would prevent unnecessary and potentially harmful bleeding of the patient if the clot capture chamber is opened to vent before the valve is closed between the catheter and the clot capture chamber. If after the pump, it does not interfere with the reservoir's blood return system, if present.

As mentioned, in some cases two parallel peristaltic pumps may be used. Generally, peristaltic pumps can be tuned to have either high flow/low pressure or low flow/high pressure (e.g., by selecting large bore tubing, and/or small bore tubing). For thrombectomy, there may be times when it is desirable to have either high flow/low pressure or low flow/high pressure. For example, when searching for the clot, the clinician may want low flow rate test aspirations to determine if the clot is near. Once they believe the clot is near, the clinician may want a high flow pulse of suction in order to engage the clot into the catheter. Once the clot is engaged and the catheter is clogged, high flow is not necessary and a high vacuum force may be desired. In some cases, the apparatus may have two pumps arranged in parallel to each other that could be operated at the same time or have could be valved to select which one is in use. When operated at the same time, during high flow conditions, the low flow/high pressure pump will not be able to keep up and will essentially be muted by the high flow pump. When there is a clog (e.g., clot is engaged) and the flow rate decreases to a level that the low flow/high pressure pump can keep up with, then the line pressure will go down to very high vacuum (low pressure) to pull the clot through (high vacuum may be considered low absolute pressure approaching full vacuum). Such a dual peristaltic pump system may be used in any of the examples described herein as the pump and may be positioned either before or after a clot capture chamber.

The tubing used in the peristaltic pump may be re-enforced. In some cases the tubing is configured for operation at either or both high flow/low pressure or low pressure/high flow. In some case the tubing may be peristaltic tubing that has Nitinol wire reinforcement. For example, Nitinol, which is capable of extreme deformation, may be used to reinforce an elastomeric tube for peristaltic tubing since and may deform down enough to seal off but can still open back up (restore) with higher force than typical elastomers. The tube may be made of an elastomeric material that can seal well. The vacuum force generated by the Nitinol forcing the diameter open rather than just the elastomer and may result in higher vacuum forces than pure elastomeric tubing for a given flow rate (inner lumen diameter).

In some examples, as described in greater detail below, the tubing used in the peristaltic pump may be placed inside an elastomeric and/or sprung sleeve. When the outer tube expands, the outer tubing may help support the inner tube preventing it from collapsing/staying collapsed. At the same time, the outer tube does not need to collapse fully, only enough to fully collapse the inner tube. Such a tube within a tube approach can be advantageous for fluid containment.

### Vacuum-Augmented Peristaltic Pump

Described herein are vacuum augmented peristaltic pumps (VAPPs), which may also be referred to herein as vacuum enclosed peristaltic pumps. These pumps may use a vacuum force (negative pressure) on the outside of the peristaltic pump tubing to provide an additional restoration force to the tubing which may enhance the pressure and/or flow rate of the peristaltic pump and/or improve the metering capabilities over a range of supply pressures.

A peristaltic pump may provide pumping action in which rollers compress the tubing and move along it, pushing fluid along as they progress. More fluid is drawn into the pump by the tube returning to its original shape after the roller passes, restoring the diameter of the tubing. The vacuum force may be dependent on the compliance/stiffness of the tube which may affect vacuum lift, output pressure, and flow rate. The vacuum augmented peristaltic pumps described herein may decouple vacuum lift from the other design constraints. This makes it possible to increase the vacuum lift of the pump/tubing set even if the tubing has little or no natural stiffness, may allow for modulation of the vacuum lift, may improve the consistency of metering (even under conditions close to vacuum lift limit or low return force tubing), may improve flow rate under high vacuum lift conditions (e.g., close to limit), and may allow for alternative tubing designs, such as naturally lay flat tubing and/or very low wall thickness tubing than previously available. These apparatuses may also allow for relatively large bore tubing to be used, which may provide high/full vacuum lift. This is an improvement over currently available systems, in which high end, large bore tubing (e.g., 0.62″) can only pull a limited amount (e.g., 24″ Hg). In general, the vacuum augmented peristaltic pumps described herein may enable improved use of tubing that does not return well to its original shape, such as plastic and/or thin walled tubing, and may provide improved efficiency as they do not require stiffer/thicker walled tubing for high vacuum lift.

For aspiration of clot from the vascular system, it may be particularly advantageous to have a high flow rate for engagement of clot, but also a high peak vacuum pressure to pull captured clot through the catheter. A traditional single peristaltic pump system may be ill suited to this use, due to the tradeoff between flow rate and vacuum lift. The vacuum augmented peristaltic pumps described herein may avoid this tradeoff and make it possible to augment the vacuum lift capabilities of tubing. These vacuum augmented peristaltic pumps may improve the efficiency of the pump, making it possible to achieve both higher flow rates and lower vacuum compared with a conventional approach.

The vacuum augmented peristaltic pumps described herein may include a sealed peristaltic pump head region in which the pressure in the pump head housing (e.g., the volume outside the tubing) can be adjusted to increase the achievable vacuum pressure or tolerable positive pressure (corresponding to negative pump head pressure or positive pump head pressure respectively). The pump head housing may be sealed such that vacuum and/or pressure is maintained. The head portion may include the rollers and tubing set used to generate the peristaltic pumping. Sealing the head portion may include enclosing the head portion in a pressured chamber (or forming a pressurizing chamber around/over the head portion).

Sealing of the head can be done via various means. In some cases the motor shaft and the tubes going into and out of the housing may include a seal to allow pressurization. As used herein, pressurization may include pulling a vacuum. In some cases the entire motor assembly, including the rollers, may be within the pressurization (e.g., vacuum) chamber; alternatively, just the rollers may be within the pressurization chamber. Numerous shaft seal methods are applicable. Similarly, the seals around the tubing (tube seals) can be made on the process tubing, on the peristaltic tubing, e.g., through a fitting, such as a bulkhead fitting, or through a combination of different sealing techniques. The walls of the chamber may be fitted with fittings, such as barbs, becoming part of the fluid path.

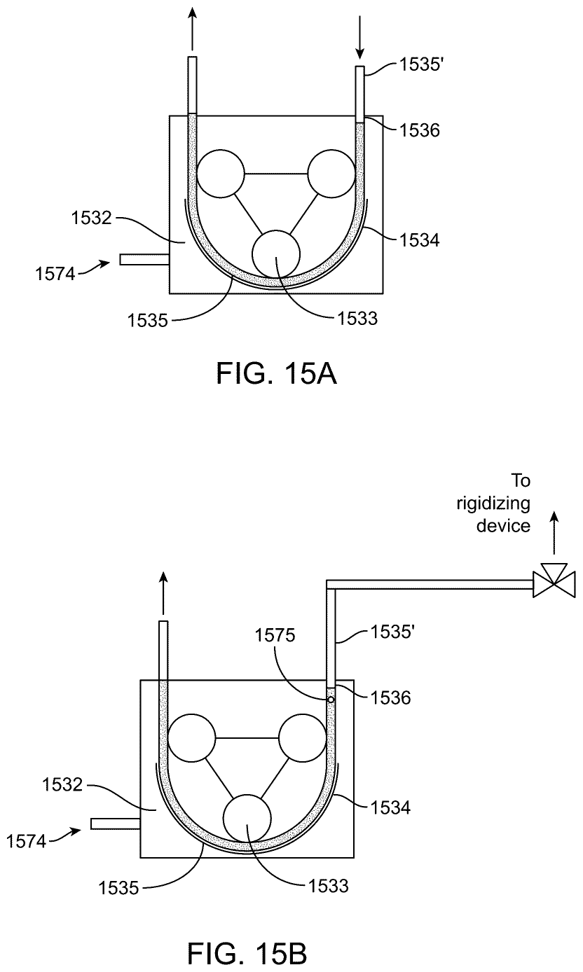

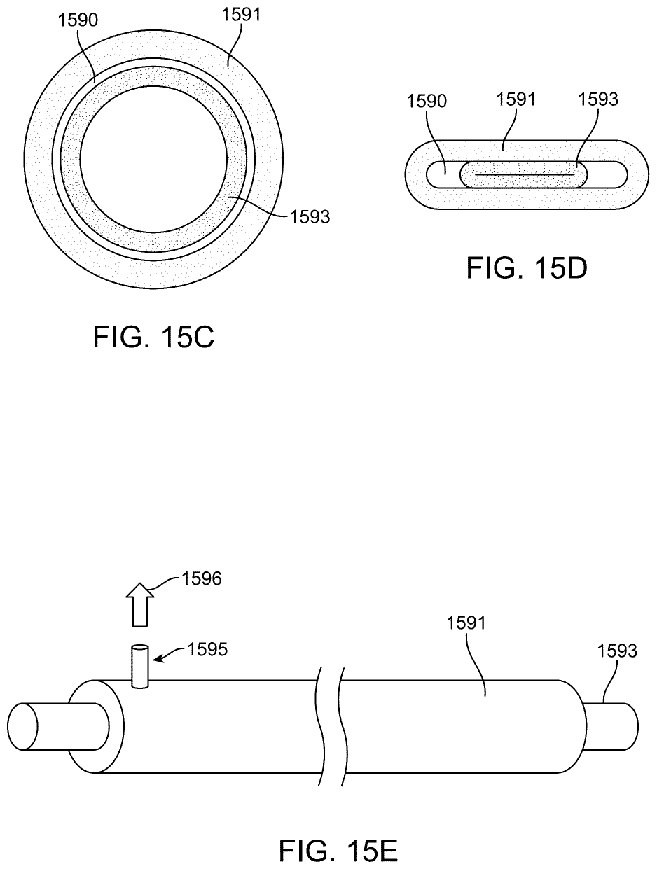

FIGS. 15A and 15B schematically illustrate examples of vacuum augmented peristaltic pumps as described herein. In FIG. 15A, the vacuum augmented peristaltic pump includes a pressurizing chamber, e.g., formed as a sealed peristaltic pump housing 1532 that encloses the tubing (e.g. peristaltic tubing 1535) and rollers 1533. One or more inlets 1574 for applying the vacuum within the sealed pressurizing chamber may be included. The rollers compress the tubing against the compression surface 1534, which may form one of the boundaries of the pressurizing chamber 1532. The tubing enters into and out of the pressurizing chamber through sealed tube ports 1536. The peristaltic tube 1535 may be continuous or coupled with the tubing 1535′ outside of the pump. In some cases the entire pump may be enclosed in the pressurizing chamber 1532, or only the had region of the pump, e.g., the tubing and rollers. The pressurizing chamber 1532 may be coupled to a source of negative pressure (e.g., a separate pump, such as a suction pump, not shown) so that the pressurizing chamber 1532 may be pressurized to a negative pressure prior and/or during use. The vacuum within the pressurizing chamber may be pressurized to any appropriate negative pressure. This pressure may be maintained during operation, and may counter any leak.

In operation, the vacuum augmented peristaltic pump apparatuses described herein may result in a significant improvement, as compared to the same peristaltic pump without applying a vacuum around the peristaltic tubing. For example, table 1, below, shows the changes to the maximum achievable vacuum and pressure rating of the peristaltic tube under standard (ambient/atmospheric) conditions, an elevated pressure, and a negative pressure.

TABLE 1

| Housing Pressure | Vacuum (PSI) | Pressure (PSI) |
|---|---|---|
| Ambient | 8 | 20 |
| Positive 5 psi | 3 | 25 |
| Negative 5 psi | 13 | 15 |

As an example under ambient conditions, a peristaltic tube may have a vacuum lift rating of 8 psi and a maximum output pressure of 20 psi. If the tubing is placed under positive pressure, the vacuum lift will go down, possibly to 3 psi, but the output pressure will increase, possibly to 25 psi. Similarly, if the head is placed under negative pressure, the vacuum lift will increase, possibly to 13 psi, while the maximum allowable output pressure will decrease, possibly to 15 psi.

The vacuum augmented peristaltic pumps described herein may pump fluid at pressures that would not normally be possible with a given piece of peristaltic tubing. For instance, as in the table above, if the peristaltic tubing has a nominal rating of 8 psi on vacuum and 20 psi on pressure, but the source was at 50 psi, the tubing/peristaltic pump could not be used to meter fluid from the source. However, if the pump housing were pressurized, e.g., to 50 psi, the peristaltic tubing/pump would function normally (provided the exit port is restricted enough to keep the pressure high enough that the housing pressure does not cause the tubing to be pushed out of the housing).

Similarly, and counterintuitively, it is possible to pump fluid even at full vacuum using the vacuum augmented peristaltic pumps described herein. If the lines are fully primed, and the source fluid is at full vacuum, bringing the peristaltic pump housing also to full vacuum means that the tubing will open. This will cause fluid to flow at the full rated flow rate provided that the fluid does not need to be lifted (i.e. you are draining a tank from the bottom, not lifting fluid out of the tank).

The vacuum augmented peristaltic pumps describe herein may therefore use thinner-walled tubing, which may be compressed more quickly/effortlessly and completely and which may restore to the uncompressed configuration more readily, assisted by the negative pressure within the pressurizing chamber. This may allow the peristaltic pump to achieve the high flow rates described above. Such thin-walled peristaltic pump tubing, without external vacuum or internal pressure would not (or would negligibly) return to its original shape to facilitate pumping. The thin-walled tubing may, in some examples, be fiber-reinforced thin walled tubing.

Although these vacuum augmented peristaltic pumps are described in the context of aspiration and systems for aspiration, these vacuum augmented peristaltic pumps may also be used for other applications, including other medical, or non-medical applications. For example, these apparatuses may be used for applying negative and/or positive pressure to including for rigidizing an apparatus, as mentioned above. In general, these vacuum augmented peristaltic pumps have enhanced vacuum capabilities of the tube/pump are increased via the vacuum augmentation inside of the inside of a sealed peristaltic pump head housing. This may enhance the natural return force in the peristaltic tubing allowing it to pull higher vacuum than either the tube or the vacuum source used to evacuate the pump head individually.

For example, these vacuum augmented peristaltic pumps may be used to control the rigidization state (e.g., rigid to flexible) of a rigidizing device including the rigidizing catheters and overtubes described herein. In such devices a bladder may compress against a rigidizing layer within the wall of the deice (e.g., extending in the tubular wall) in order to rigidize the device. This compression locks the rigidizing layer in place, making the device more rigid. The rigidizing device may include a plurality of lengths of filament that cross over each other and that may slide freely over each other in the flexible state, but when compressed by the bladder layer, are prevented from sliding and may therefore rigidize the device. Typically, the greater the applied pressure (either negative pressure between the rigidizing layer and the bladder and/or positive pressure on the bladder to drive the bladder against the rigidizing layer), the greater the stiffness.

Since the vacuum augmented peristaltic pumps described herein are used primarily to pull vacuum, the vacuum port of the vacuum augmented peristaltic pumps can also or alternatively be connected to the pump housing. In some configurations, this can be accomplished via a leak in the tubing before the rollers. For example, the vacuum applied to the peristaltic pump may be used to provide negative pressure for rigidizing the rigidizing tool. The pressurized chamber comprising the peristaltic tubing, rollers, and housing, may be connected in-line with a valve that controls the ridigization state of the rigidizing. In some cases the positive or negative pressure applied to rigidize the tool may come from the input/output of the peristaltic pump. The vacuum on the inside and outside of the peristaltic tube are equal, the tube will always return to its original shape.

For clot aspiration, the vacuum augmented peristaltic pumps described herein may provide both high flow for clot engagement and high vacuum pressure to pull the clot down the tubing. High flow rate (e.g., greater than 1.0 L/min or more, such as 1.5 L/min or more, 2.0 L/min or more, etc.) may be achieved in any of these systems with a large ID peristaltic tube, and a high return force may concurrently be achieved due to the enhancement of the tubes natural return force.

One possible variation of the vacuum augmented peristaltic pump may place the inner peristaltic tubing 1593 inside a larger tube 1591, as shown in FIGS. 15C-15E. In this example, an outer tube 1591 may have enough return force to return to circular when the ID 1590 is under negative pressure and is crushed by the rollers of the peristaltic pump. By evacuating the space between the interior peristaltic tubing and the exterior support tubing, all the gains realized for the pump head can be realized. The outer tubing only need crush (shown in FIG. 15D) sufficiently to fully close the interior peristaltic tubing. FIG. 15E shows an example of a length of such an assembly, including a pressure connector 1595 for applying negative pressure 1596 within the space between the tubing. This configuration has some advantages. The pump head no longer needs to be sealed. This allows for quick exchanges of tubing. This may use the tubing as a single use component than replacing the entire pump. It also allows for containment of fluid should the peristaltic tubing rupture, both the internal and external tubing can be controlled cleanliness and materials. The outer tube can also limit the expansion of the inner tube, allowing for much higher outlet pressures. The outer tube may be reinforced to further augment outlet pressures.

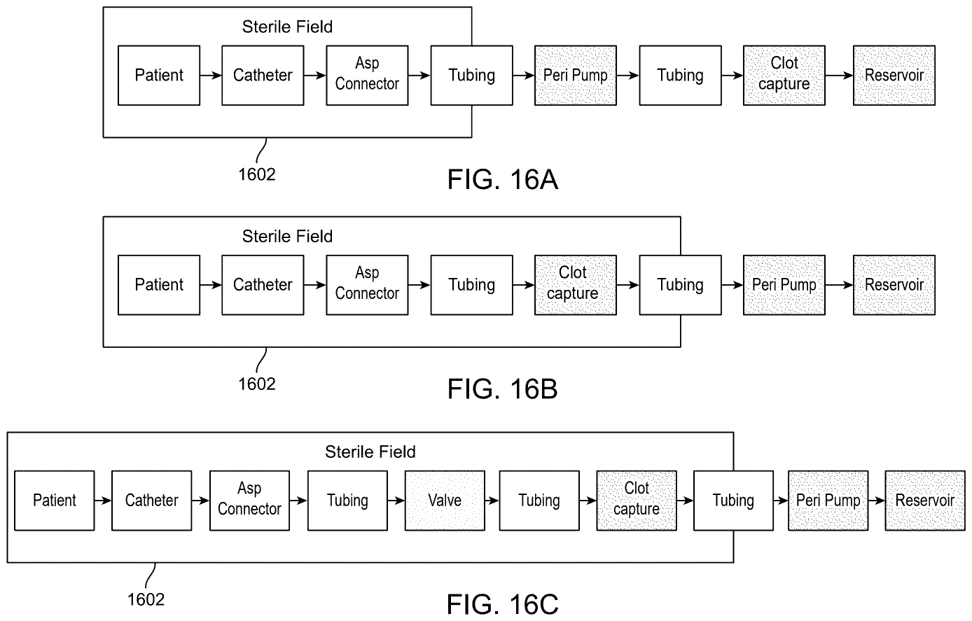

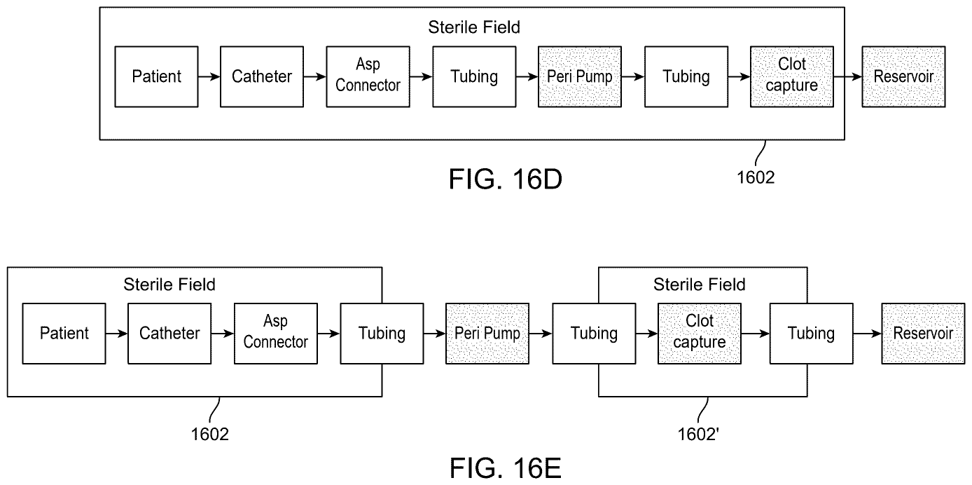

The vacuum augmented peristaltic pumps described herein are also advantageous because the fluid path is fully controlled/contained; this path may be hemoprotective as hemolysis is generally associated with high shear and exposure to air/bubbles. Further, filtering may be performed before or after the pump stage, since clot material can pass through the vacuum augmented peristaltic pump without damaging the pump, or getting torn into a bunch of pieces. These vacuum augmented peristaltic pumps may be fully or partially configured as single-use or multiple use pumps, and may be configured to be used within the sterile field or outside of the sterile filed. Various configurations showing examples of these different configurations are shown in FIGS. 16A-16E. In general, these methods and apparatuses may include a catheter (e.g., an aspiration catheter and/or an outer rigidizing catheter), as aspiration line (e.g., tubing), a peristaltic pump (“peri pump”) an optional clot capture chamber (“clot capture”), a reservoir for holding aspirated blood (“reservoir”), and one or more valves (e.g., for blocking/occluding the aspiration line or other lines (tubing). The system may also optionally include one or more return catheters and/or return tubing. In some cases a separate pump (syringe, peristaltic pump, etc.) may be used to return blood to the body.

Any of the apparatuses described herein may include a control system (control sub-system) that may be configured to operate a control algorithm controlling operation of the peristaltic pump (e.g., the multiple pump heads, and/or VAPP). For example, these methods or apparatuses may control the pump to deliver: high flow then low flow, and/or low flow then high flow, and/or may include a low flow setting and/or high flow setting, may be configured to provide one or more of: variable flow control, high flow pulses, simple full on/full off, and/or timed pulses.

In FIG. 16A, the schematically illustrates an example in which clot material and blood is aspirated and collected. The system include an aspiration catheter that is connected to a tubing (e.g., aspiration line) by an aspiration connector, and the tubing fluidly connects the aspiration catheter to the peristaltic pump; proximal to the pump the tubing is also connected to a clot capture chamber and a reservoir. In this example, the portion of the system within the sterile surgical field is shown by the box 1602, and includes the patient, aspiration catheter, connector and part of the tubing.

Another example is shown in FIG. 16B, and includes the aspiration catheter that is connected to the tubing (e.g., aspiration line) by an aspiration connector and to a clot capture chamber that is distal to the peristaltic pump; proximal to the pump the tubing is also connected to a reservoir. In this example the sterile field 1602 includes the patient, catheter, the clot capture chamber and a portion of the aspiration tubing.

FIG. 16C shows an example that is similar to that shown in FIG. 16B, but with a valve between the catheter and the clot capture chamber. The valve may be opened or closed depending on whether the pump is on (e.g., suction is being applied) or not. Thus, the system includes the aspiration catheter connected to the tubing (e.g., aspiration line) by an aspiration connector and to a clot capture chamber via a valved region of the tubing. Both the valve and the clot capture chamber are positioned distal to the peristaltic pump; proximally to the pump the tubing is also connected to a reservoir. The sterile field 1602 include the patient, catheter, connector, valve, clot capture chamber and a portion of the tubing connecting these.

In FIG. 16D the peristaltic pump is also included in the sterile filed 1602, which also includes components between the patient and the peristaltic pump (e.g., the catheter, connector, peristaltic pump and clot capture, as well as a portion of the tubing. The reservoir is outside of the sterile field in this example. Also in this example, the clot capture is on the opposite side of the peristaltic pump as compared to other variations. This allows the peristaltic pump to be as close to the catheter as possible to reduce issues with priming, air entry when the clot capture chamber is opened, and reduce blood loss.

Finally FIG. 16E shows an example in which the pump and reservoir are outside of the sterile filed 1602 but the rest of the system is within the sterile field. This configuration may be possible because the peristaltic pump allows the material inside of the tubing to remain sterile within the tubing, which does not need to be opened or exposed to atmosphere to pump. In some examples, the peristaltic pump may be connected to a valve by a large bore tube, which may be substantially larger than the tubing connecting the valve to the catheter. The peristaltic pump may remain on even with the valve is closed. Thus, vacuum may be generated within the volume of the tubing before the valve is opened. A peristaltic pump with a high vacuum but low flow rate may be used. In this case, the valve may remain closed for a certain period of time in order for the vacuum level to reached the desired level (e.g., for 3 seconds). When the valve is opened, immediate and short term high vacuum flow is achieved, but the pump cannot keep up the flow rate to maintain high flow. This allows for quick engagement of the clot without long term high flow which can cause high blood loss if the clot is not engaged. If clot is engaged then the pump is able to keep high vacuum in the low flow condition to pull the clot through. There does not need to be filtration between the valve and the peristaltic pump, since the peristaltic pump can accept the thick clot and will simply chop it as the clot passes the rollers. The clot will be chopped into a segment determined by the spacing of the rollers.

### Blood Return Sub-Systems

In general, any of these apparatuses may include a reservoir of other blood collection element, which may, in some cases, be configured to provide a passive return force. For example, the apparatus may include an elastic or elastomeric bag and/or a weighted/sprung syringe, e.g., an elevated bag/gravity dispense, including a closed vessel that may result in an air spring to drive blood back into the body. For example, when a blood bag is used, the blood bag can reinfuse patient using standard procedures. Alternatively, the reservoir may be maintained at some positive pressure (as via an clean dry air source or otherwise) and the positive displacement pump overcomes that pressure.

In some examples, filtered or partially or non-filtered blood may be driven into an elastomeric bag or a weighted/sprung syringe. Both of these designs are able to increase in volume as more filtered blood is added, and may act as a reservoir of blood that may then apply pressure to force the blood back into the patient when desired. These are passive devices. They do not need to be activated in anyway; blood simply goes in and comes out because of the existing force stored by the system (e.g., either due to gravity or by loading the spring force in the system). This type of pressure charging is enabled by a peristaltic pump rather than a vacuum pump. In some cases the system may include a valve in between the passive reservoir(s) and the second catheter used for returning blood to the patient. When the valve is opened, the filtered blood is pushed into the patient by the pressure created by the reservoir. The advantage to this is that the user does not need to perform any actions other than opening the valve (and monitoring blood flow to ensure no air is infused). The pressure of reinfusion is controlled by design and not by the user to reduce the risk of overpressure or too quickly reinfusing the blood. Additionally, the user does not need to manage reinfusion. The valve could be left open throughout the procedure and reinfusion would be done automatically without any user intervention whenever aspiration is performed. The weighted syringe concept ensure perfectly consistent pressure. Unlike an elastomeric bladder or other spring type of force application, a weight provides a consistent weight assuming it exists on Earth and is setup vertically whereas the force for a spring loaded system or elastomeric system would change depending on how much the spring or elastomer is deformed.

The peristaltic pump may also act as a hub for infusion related monitoring such as bubble/air detection to further reducing operator burden. In any of these apparatuses and methods, automated reinfusion may include a chamber with a pressure activated/relief valve between the chamber and the atmosphere. The valve may be in the closed position and may open to atmosphere when the internal pressure of the chamber is some predetermined amount based on a safe reinfusion pressure to the patient. A valve between the chamber and the patient can then be opened to reinfuse blood back into the patient.

Any of the apparatuses and methods described herein may include a blood return (e.g., blood reinfusion) sub-system, which in some cases may include an air capture system that is in-line with the reinfusion tubing to eliminate/prevent air going into the body.

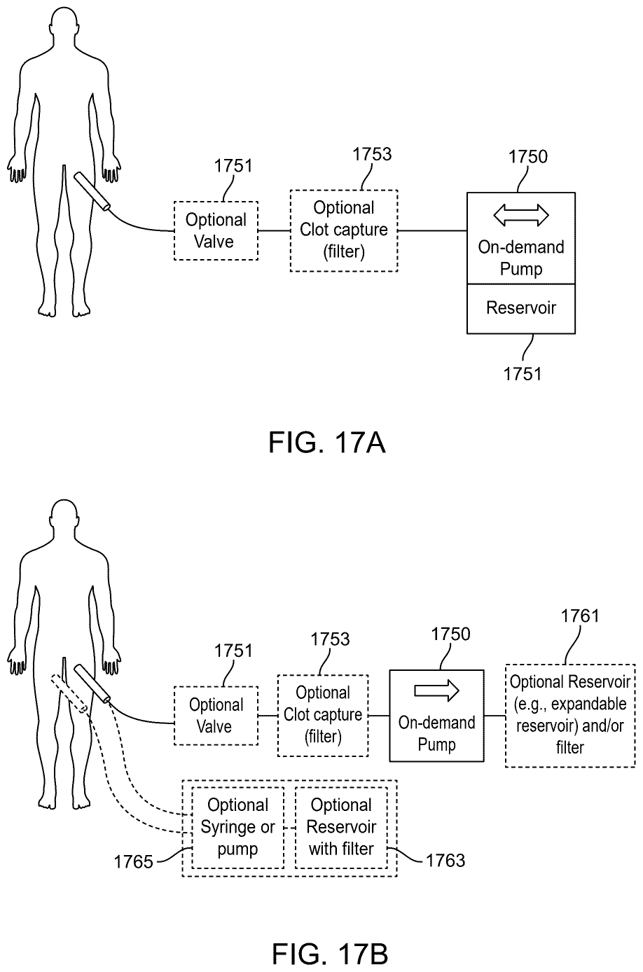

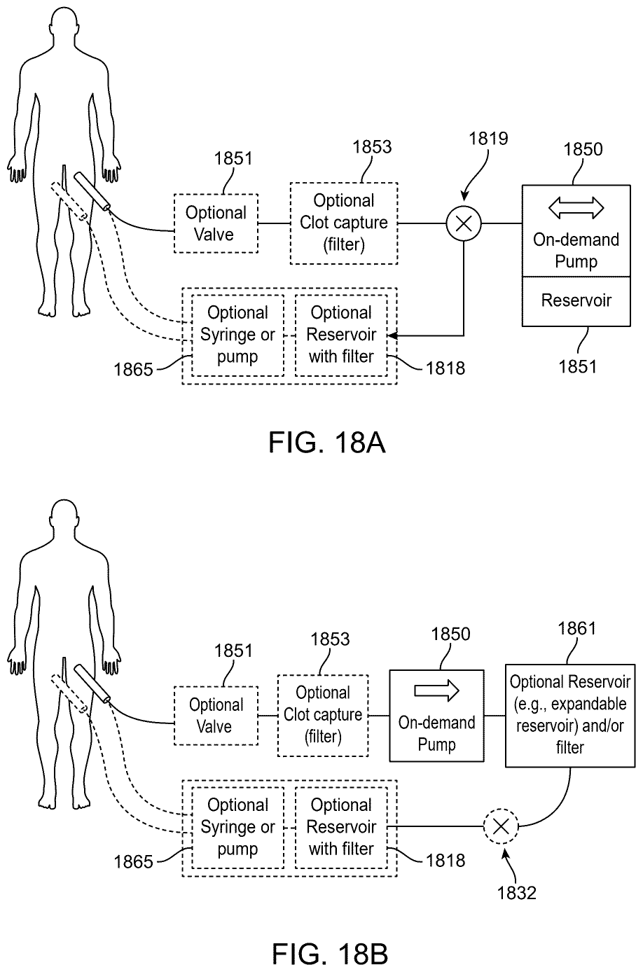

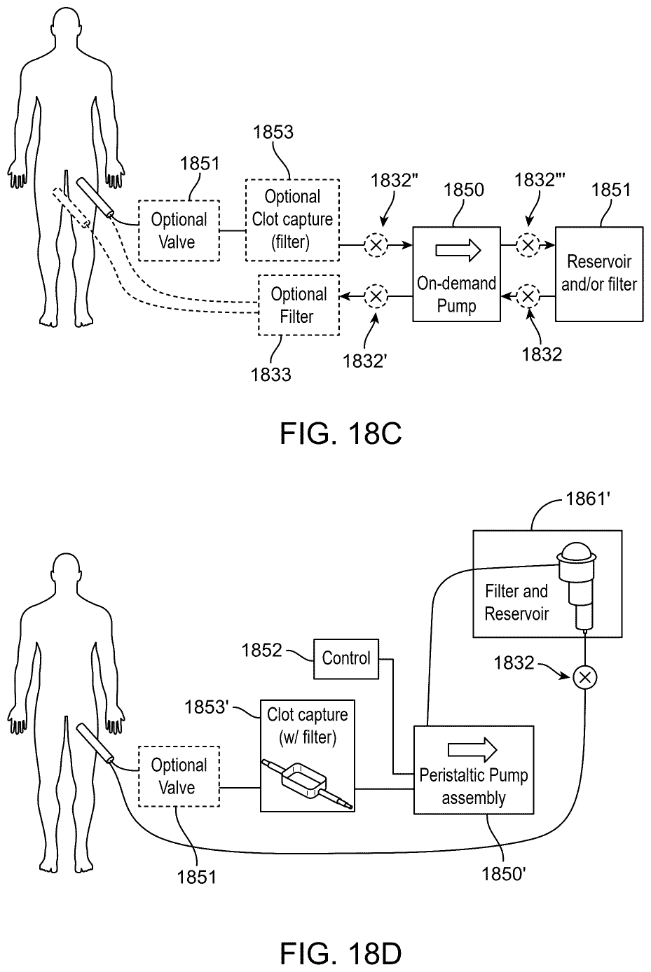

FIGS. 17A-17B and 18A-18C schematically illustrate various systems for removing clot material using a peristaltic pump, or more generically, an on-demand positive displacement pump; FIGS. 18A-18C shows systems with automatic or semi-automatic blood return, and may include blood-return sub-systems. For example, FIG. 17A shows a generic configuration with an on-demand pump 1750 (e.g., any of the peristaltic pump apparatuses described herein), and an optional valve 1751 and clot capture chamber 1753. The system may also include a reservoir 1751 (e.g., blood collection reservoir). The system may also include an integrated pump switch that may be coupled to the valve 1751, e.g., so that when the pump is off (not aspirating) the valve is closed, preventing bleeding from the patient. Any of these systems may include a user controlled switch (control) that turns the peristaltic pump on and off. In some cases the valve may have a user controlled switch to turn the pump on/off that may also act as a fluid control valve.

FIG. 17B shows a system that does include blood return, but the blood return is not part of the same blood circuit. In this example the system may include a reservoir 1763, which may include a filter, and a pump for pumping the blood back into the patient, e.g., through a second catheter. Thus, the system may include a catheter (e.g., aspiration catheter and/or outer rigidizing catheter) a valve (and/or an integrated pump switch) 1751, a clot capture chamber 1753, a peristaltic pump 1750 and a reservoir 1761. In some cases the reservoir may be an expandable reservoir, for passive return of the blood the patient, as described above. Any of these systems may include a filter, e.g., in some cases as part of the reservoir, in-line, or elsewhere coupled to the aspiration tubing. The filter may be, for example, a 40 micron filter. The blood return sub-system may include a second (optional reservoir) 1763 and a second pump 1765 (e.g., syringe pump, peristaltic pump, etc.). In some case the syringe may be both the pump and the second reservoir.

FIG. 18A shows an example of a system including a peristaltic pump 1850 and an integrated blood return sub-system (e.g., option secondary reservoir and/or filer 1818, and secondary pump). In this example the system may include the aspiration catheter, a valve 1851 (e.g., integrated pump switch), clot capture chamber 1853, on-demand pump (e.g., peristaltic pump) and a first reservoir 1851. The reservoir maybe coupled to a two-way valve 1819 to allow the pump to drive blood either into the reservoir (during aspiration from the body) or to switch direction (and switch the 2-way valve) to drive fluid from the first reservoir into the blood return circuit and either directly into the body or into a secondary reservoir 1818 where it may later be pumped into the body, e.g., using a secondary pump 1865.

FIG. 18B shows a similar example, in which the blood may passively or actively be driven from the first reservoir (e.g., collection chamber) 1861 into a second reservoir 1818 or directly into the body, using a valve 1832.

In any of these examples the peristaltic pump may be used as a pump to both remove (aspirate) blood and to return blood back into the body. For example, FIG. 18C shows a variation in which a series of valves 1832, 1832′, 1832″, 1832″″ can be controlled to coordinate blood flow from the aspiration catheter through an optional clot capture device (with filter) 1853, to be stored in a reservoir 1851 (optionally filtered) and one or more valves 1832 may control delivery, using the same peristaltic pump 1850, into the body though a second catheter, either directly or after further filtering (e.g. in-line filter 1833).

FIG. 18D shows an example of a system including a peristaltic pump apparatus as described herein, such as (but not limited to) a peristaltic pump apparatus having two or more pump heads (that may be configured to operate out-of-phase). In FIG. 18D, the system (as in any of the examples shown herein) may optionally include a catheter (e.g., clot removal catheter) The system may include a fluid line with an optional valve 1851, and in the example shown, a clot capture chamber having a filter 1853′ that is coupled distally of the peristaltic pump assembly 1850. A control 1852 on the peristaltic pump assembly or communicating with the pump assembly may control operation of the pump. The pump in this case may pass the blood on to a filter and second chamber 1861′ that may be elevated relative to the patient. A valve 1852 may be used to turn on/off blood return to the patient (e.g., to the sheath used with the aspiration catheter). The blood return reservoir 1861 may be pressurized with air or other gas, to assist the gravity feed of the blood return (e.g., a manual or automatic pump may be used).

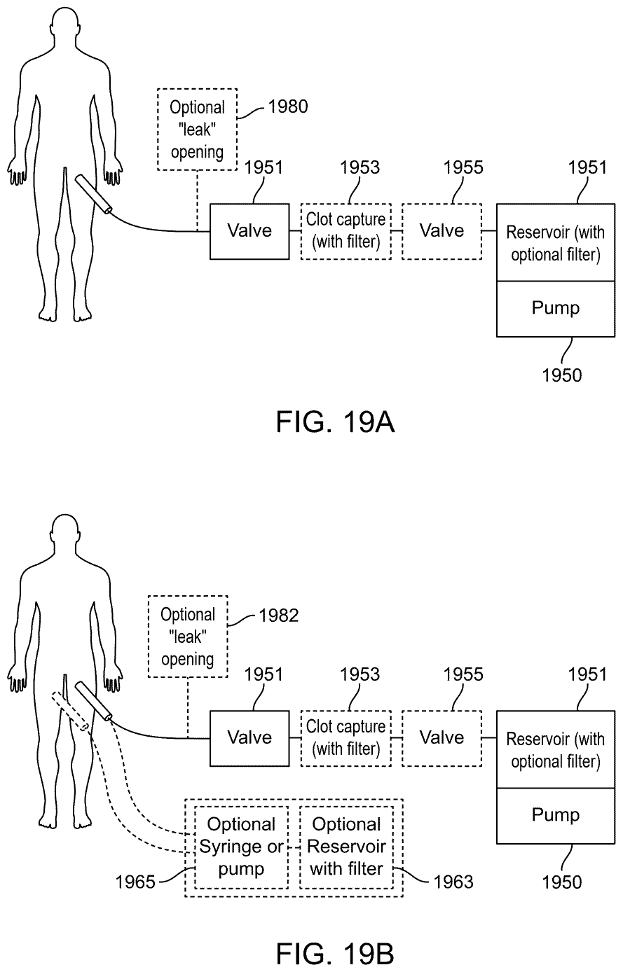

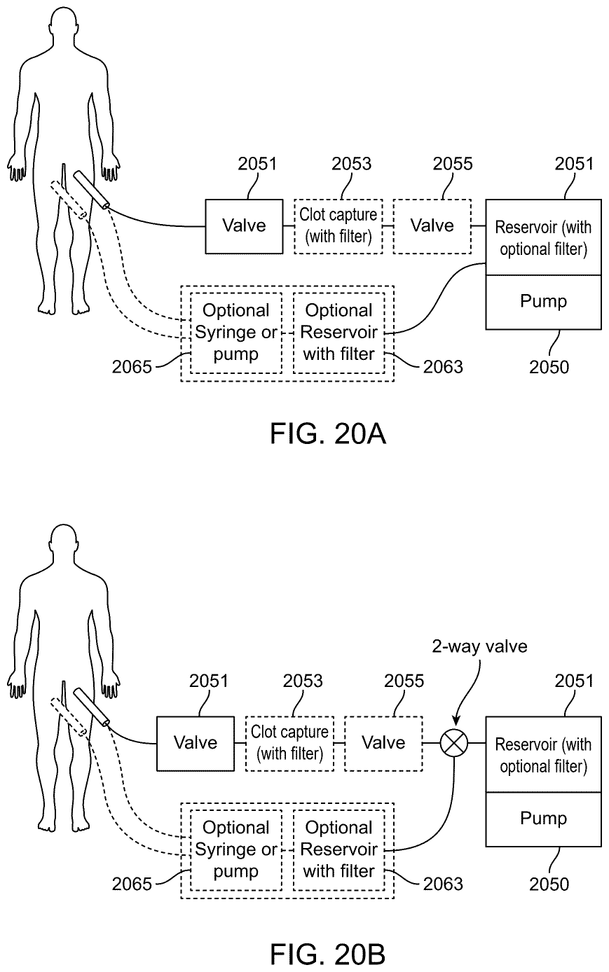

FIGS. 19A-19B and 20A-20B illustrate other alternative blood circuits and systems. In FIG. 19A the pump is not necessarily an on-demand pump (e.g., peristaltic pump) but may be a more traditional pump. In this example, pressure may be stored in the pump 1950 or in the reservoir 1951 and/or the clot capture chamber 1953 and released by opening the valve 1955 or 1951. In some cases the suction pressure is not stored, but suction may be continuously applied, but diverted from the body by an optional leak opening 1980 as shown in FIG. 19B. In this case the pump may be run continuously, but aspiration will not be applied to the body until a valve 1951 is opened top place the catheter in fluid communication with the aspiration line, while closing the leak path 1982. Blood may stop flowing by releasing the covering in the leak and simultaneously or subsequently closing the valve to the aspiration catheter. In FIGS. 20A and 20B the blood return sub-systems are integrated into the system. For example, FIG. 20A shows a system with an integrated blood return sub-system. In some cases the pump may be turned on/off rather than building up negative pressure before opening the valve.

In general, in any of FIGS. 17A-17B, 18A-18C, 19A-19B and 20A-20B, the various combination of pumps, valves, filters, chambers and tubing may be arranged in any appropriate order, including but not limited to those explicitly described above.

### Example: Treating Acute Pulmonary Embolism

As mentioned above, any of these methods and apparatuses may include the use of a rigidizing catheter either as the aspiration catheter or in combination with an aspiration catheter. For example, any of these methods and apparatuses may include the use of a rigidizing catheter as an “endo portal” that includes the use of a rigidizing outer catheter to form a stable base from the additional catheters, such as a flexible (and in some cases distally biased) inner catheter, to precisely and accurately control direct access for endovascular interventions including, but not limited to pulmonary embolectomy. These methods may significantly reduce right heart tension in acute pulmonary embolism.

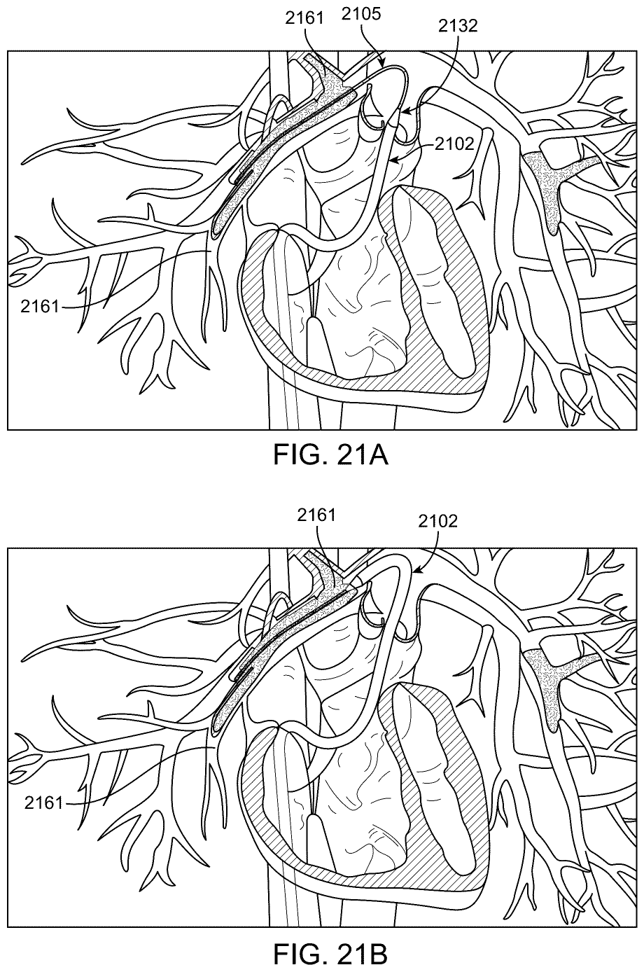

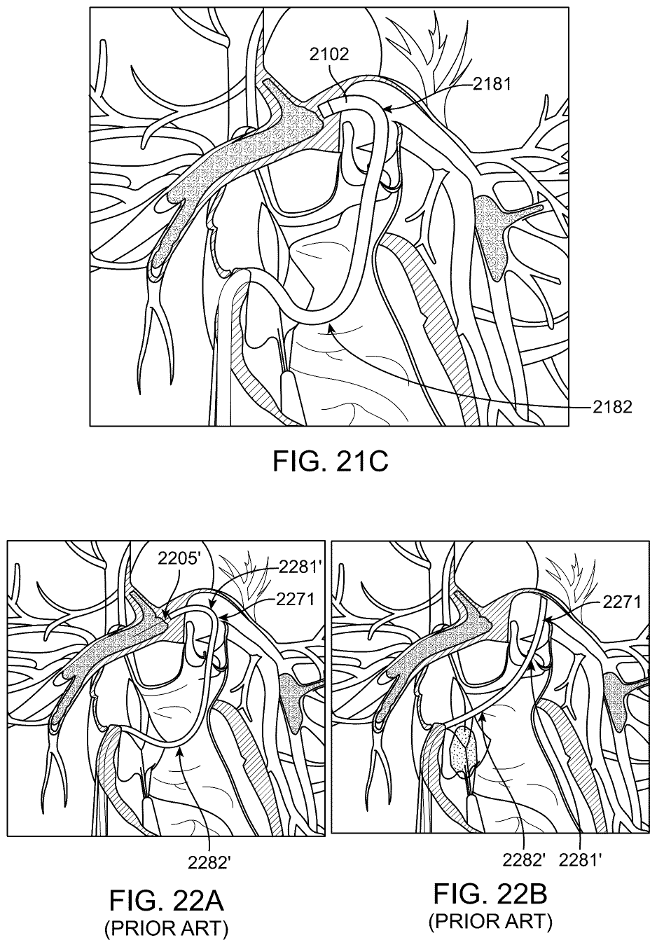

For example, these methods may include a procedure in which the outer rigidizing catheter into the pulmonary arteries. For example, the rigidizing catheter may be navigated over a guidewire (e.g., 3 French or less Nitinol wire, e.g., an 0.035 guidewire) into the pulmonary arteries. The outer rigidizing catheter may be navigated over a highly flexible guide wire in the flexible configuration. In some examples an obturator, and in particular, an obturator having an effective stiffness of less than about 2 lb-in² (such as those described above), may be inserted into the lumen of the outer rigidizing catheter to assist in tracking over the highly flexible guidewire, without significantly reducing the flexibility of the outer rigidizing catheter. FIG. 21A shows an example of an outer rigidizing catheter 2102 including an obturator 2132 riding over a highly flexible (e.g. ‘soft’) guidewire 2105 that has been positioned into an occluded region 2161 of the pulmonary arteries 2160.

Once positioned in the pulmonary arteries, the outer rigidizing catheter may be rigidized, e.g., by the application of pressure (e.g., positive and/or negative pressure). In some examples the outer rigidizing catheter may be rigidized by applying a positive pressure from a liquid, such as saline, e.g., between 2-10 atmospheres (e.g., between 4-8 atm, between 5-7 atm., greater than 3 atm, greater than 4 atm, greater than 5 atm, etc.). In some cases the outer rigidizing catheter may be positioned near or even adjacent to the occlusive clot material 2161, as shown in FIG. 21B.

Rigidizing the outer rigidizing catheter may fix the vertex outer rigidizing catheter into the shape and position that that the flexible guidewire was in, creating an endo portal. This is illustrated in FIG. 21C. The rigidized outer rigidizing catheter may provide stability and resist prolapse. For example in the rigid configuration the outer rigidizing catheter 2102 may maintain even relative tight bends 2181, 2182 that may otherwise tend to relax, and cause pressure against the vasculature in a way that may be harmful to the vasculature, including in dilated regions of the anatomy through which it passes, such as the right ventricle. The outer rigidizing catheter in the rigid state may even without a guide wire, without straightening out.

In contrast, convention catheters that do not rigidizing (or in some cases that does not rigidize to the same degree), will apply force against the heart and vasculature, as illustrated in FIGS. 22A and 22B. FIG. 22A shows a conventional catheter positioned over a guidewire similar to the method described above, prior to removing the guidewire 2105′. In this case, the conventional catheter 2271 navigates the tortious path taken by the guidewire, but once the guidewire is removed, as shown in FIG. 22B, the conventional catheter 2271 will prolapse against the heart and vessel, and cannot maintain the more tightly bent regions 2281′, 2282′. Thus, the use of the outer rigidizing catheter may both provide a stable base for aspirating, either directly from the outer rigidizing catheter and/or from an aspiration catheter inserted through the outer rigidizing catheter, and may also prevent strain and/or damage to the heart and vasculature.

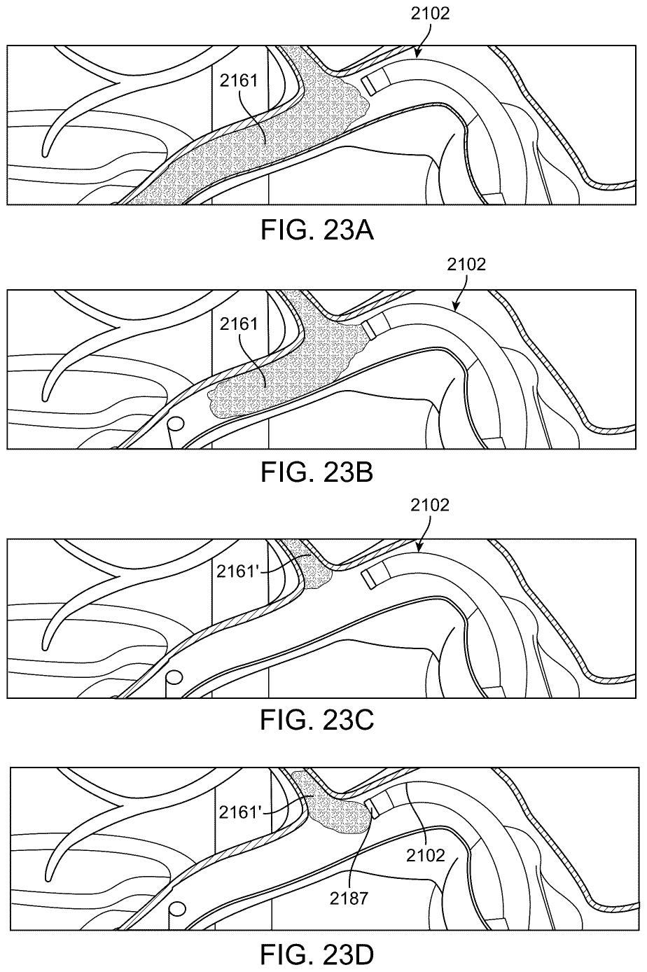

FIGS. 23A-23D illustrate the removal of clot material from the pulmonary arteries by aspirating through the outer rigidizing catheter 2102. In FIG. 23A, which shows a slightly enlarged view of FIG. 21C, the outer rigidizing catheter 2102 is positioned adjacent to a clot material (and near a bifurcation in the pulmonary artery). Aspiration may be applied through the outer rigidizing catheter while it is held in the rigid state. The outer rigidizing catheter may be connected at the proximal end (e.g., a hub) of the outer rigidizing catheter, and aspiration may be applied through the lumen of the outer rigidizing catheter to remove clot material, as shown in FIGS. 23B and 23C. Aspiration may be pulsed. The amount of aspiration may be constant or variable. In some cases the clot material may be collected into a clot capture device, as described above. For example, a control (e.g., a vacuum activation valve) may be used to turn on aspiration, including in short (e.g., 10 seconds or less, 5 seconds or less, 4 seconds or less, 3 seconds or less, 2 seconds or less, 1 second or less, etc.) pulses.

Advantageously, the outer rigidizing catheter in the rigid configuration may maintain the distal end of the outer rigidizing catheter within the lumen of the vessel (e.g., centered 2187 in the vessel lumen) preventing the distal end from suctioning onto the vessel wall even when a relatively high suction is applied through the outer rigidizing catheter, which may be a significant problem for contemporary aspiration catheters. Captured clot may be present in the clot capture chamber (as described above), and may be viewed.

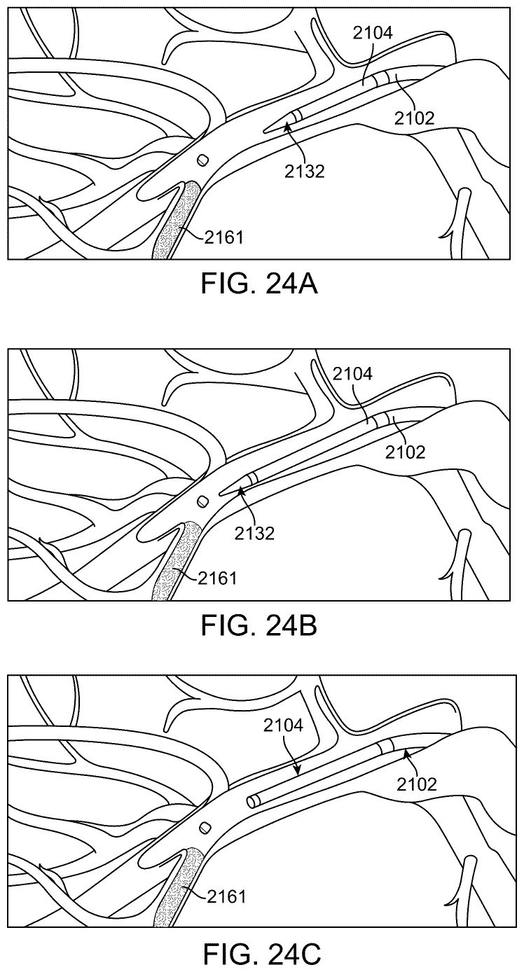

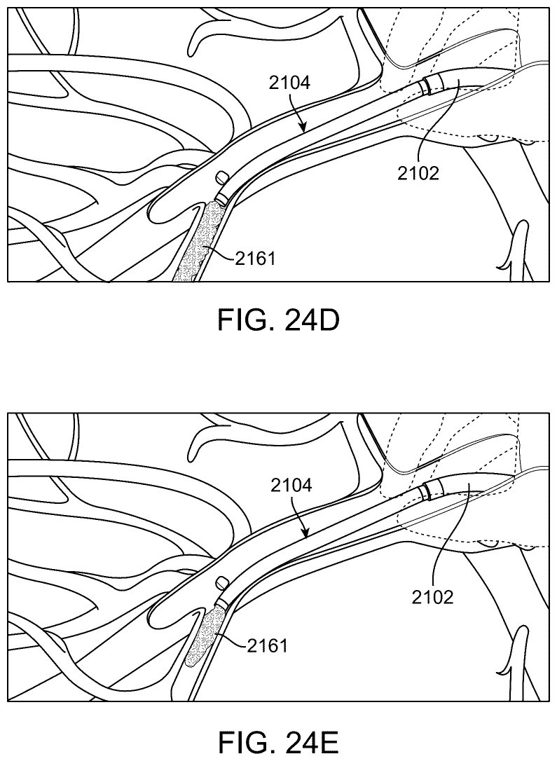

Additional aspiration may be used to remove clot material 2160′, including from branched regions as shown in FIGS. 23C and 23D. Alternatively or additionally, suction catheter may be used by inserting through the lumen of the outer rigidizing catheter, including in particular suction catheters having a distal end region that is pre-bent or biased (e.g., at an angle of between about 20-70 degrees (e.g., between about 25-55 degrees, etc.). For example, FIGS. 24A-24E illustrate the use of a rigid outer rigidizing catheter 2102 with a flexible and pre-bent inner aspiration catheter 2104, as shown. In FIGS. 24A-24B the inner aspiration catheter 2104 is advanced with a soft obturator within the lumen of the aspiration catheter through and distally out of the rigid outer rigidizing catheter 2102. The aspiration opening at a distal end region of the aspiration catheter may be positioned near the clot material 2161. In FIG. 24C the obturator within the aspiration catheter may be removed, allowing the biased distal end of the aspiration catheter to assume the pre-bent shape (as shown in FIG. 24D) so that the aspiration opening may be positioned near the clot material 2161. The source of suction may be coupled to the proximal end of the aspiration catheter (e.g., proximal hub) and aspiration may be applied through the lumen of the aspiration catheter to remove clot material, as shown in FIG. 24E.

In general, the methods and apparatuses described herein, including an outer rigidizing catheter and an aspiration catheter, may be used with similar degree of control that is possible with direct access, allowing precise positioning and holding of the aspiration catheter from the very proximal end, while manipulating and positioning the aspiration catheter distally out of the outer rigidizing catheter. The outer rigidizing catheter in the rigid configuration may prevent buckling or deflection of the aspiration catheter along its length even over extremely long pathways through the body, essentially constraining movement of the aspiration to allow just advancing/retracting and rotation (e.g., torquing) of the aspiration catheter over the region that is within the outer rigidizing catheter, from an insertion site, e.g., a femoral insertion site, to the distal end opening of the rigid outer rigidizing catheter.

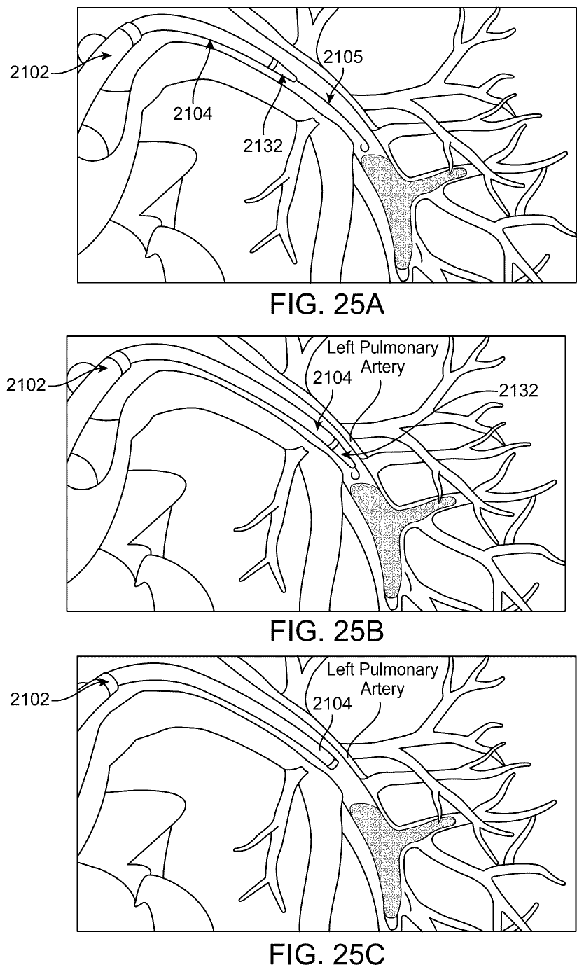

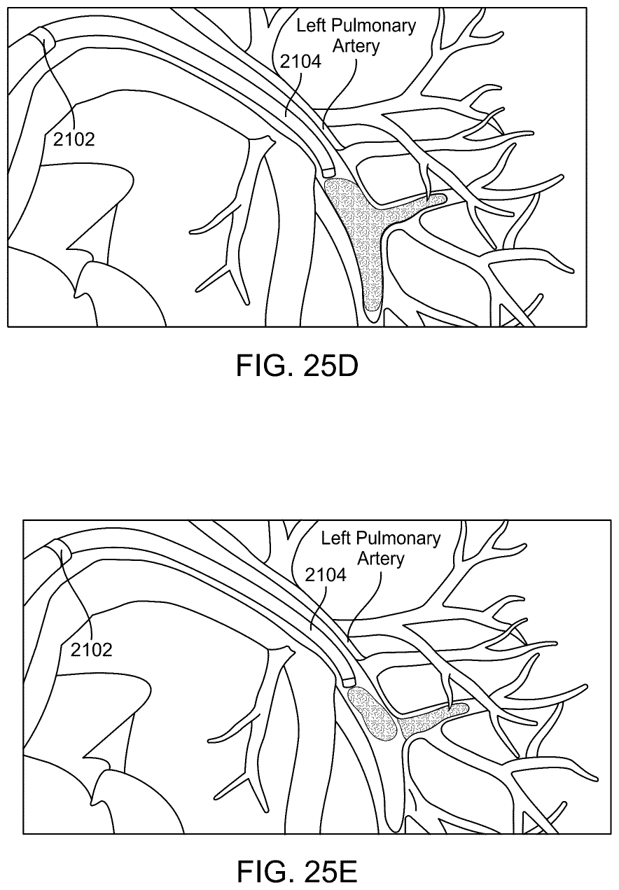

The outer rigidizing catheter may be repositioned between the left and right pulmonary arteries. For example, FIGS. 25A-25E illustrate the use of the apparatus in the left pulmonary artery after first advancing the outer rigidizing catheter 2102 in a highly flexible configuration over a guidewire, as described above in FIGS. 21A-21C. The outer rigidizing catheter 2102 may be positioned in the left pulmonary artery at a desired depth, e.g., with a soft obturator 2132 within the outer rigidizing catheter while it is advanced over the guidewire 2105. Once positioned, the outer rigidizing catheter may be rigidized by applying positive or negative pressure, e.g., using pressurized saline in some cases. Once rigid, the inner aspiration catheter 2104 with an internal obturator may be positioned within the pulmonary artery, as shown in FIGS. 25A-25B. The inner aspiration catheter 2104 may be further steered and advanced by removing the inner obturator and guidewire, as shown in FIG. 25C so that the biased (e.g., pre-bent) distal tip of the aspiration catheter may assume a bent shape, which may allow directional steering from the proximal end of the apparatus, as shown in FIGS. 25C-25D. Once the aspiration opening of the aspiration catheter is positioned, suction (aspiration) may be applied, as shown in FIG. 25E, withdrawing the clot material into the clot capture device, as described above.

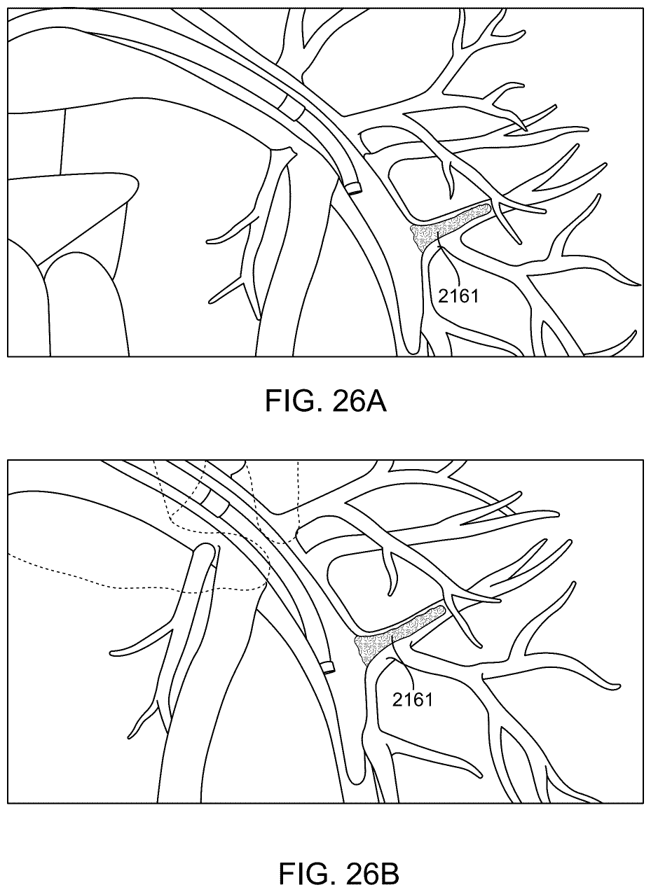

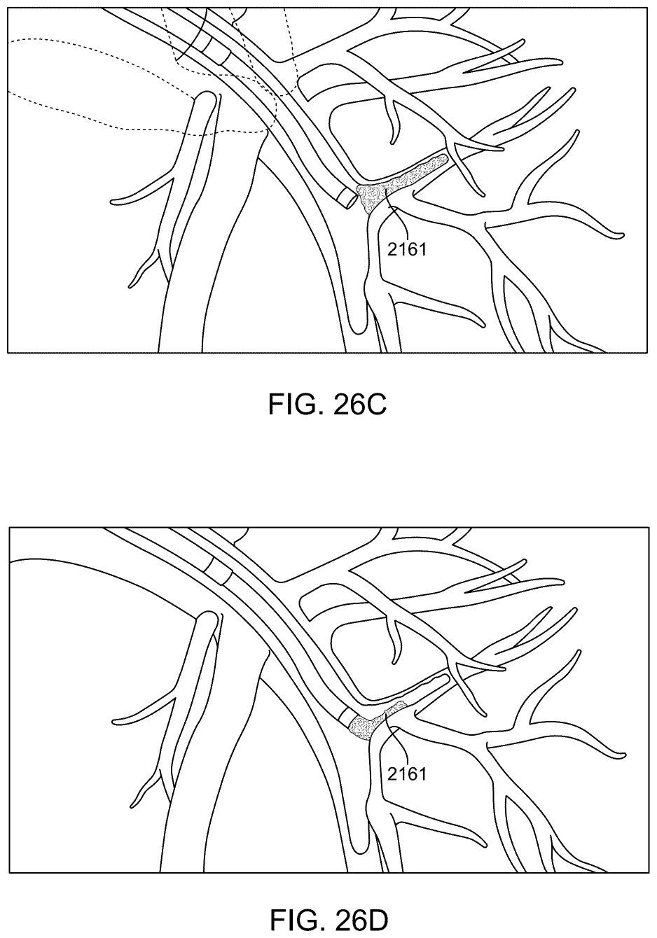

The position and orientation of the inner aspiration catheter 2104 may be precisely adjusted to engage with the clot material and navigate the vessels. The outer rigidizing catheter 2102 may be advanced further (with or without the use of a guidewire) during the procedure, as shown in FIGS. 26A-26D. For example, if lateral navigation is needed, the outer rigidizing catheter 2102 may be fixed beyond the initial curve of the left pulmonary artery by de-rigidizing the outer rigidizing catheter 2102, converting it to a highly flexible state, e.g., by adjusting the applied pressure (or in some cases removing the positive or negative pressure, or in some cases reversing the positive or negative pressure), and advancing the outer rigidizing catheter 2102 over the inner aspiration catheter, as shown in FIG. 26A. Once the outer rigidizing catheter 2102 is positioned beyond the curve, it may again be rigidized and the inner aspiration catheter may be further advanced, rotated (torqued) or retracted, as shown in FIGS. 26B-26C. The aspiration opening of the inner aspiration catheter 2104 may be positioned near (e.g., adjacent to or against) the clot material 2161, and aspiration may be applied through the aspiration catheter, as shown in FIG. 26D, to remove the clot material.

It should be appreciated that all combinations of the foregoing concepts and additional concepts discussed in greater detail below (provided such concepts are not mutually inconsistent) are contemplated as being part of the inventive subject matter disclosed herein and may be used to achieve the benefits described herein.

The process parameters and sequence of steps described and/or illustrated herein are given by way of example only and can be varied as desired. For example, while the steps illustrated and/or described herein may be shown or discussed in a particular order, these steps do not necessarily need to be performed in the order illustrated or discussed. The various example methods described and/or illustrated herein may also omit one or more of the steps described or illustrated herein or include additional steps in addition to those disclosed.

A person of ordinary skill in the art will recognize that any process or method disclosed herein can be modified in many ways. The process parameters and sequence of the steps described and/or illustrated herein are given by way of example only and can be varied as desired. For example, while the steps illustrated and/or described herein may be shown or discussed in a particular order, these steps do not necessarily need to be performed in the order illustrated or discussed.

The various exemplary methods described and/or illustrated herein may also omit one or more of the steps described or illustrated herein or comprise additional steps in addition to those disclosed. Further, a step of any method as disclosed herein can be combined with any one or more steps of any other method as disclosed herein.

When a feature or element is herein referred to as being “on” another feature or element, it can be directly on the other feature or element or intervening features and/or elements may also be present. In contrast, when a feature or element is referred to as being “directly on” another feature or element, there are no intervening features or elements present. It will also be understood that, when a feature or element is referred to as being “connected”, “attached” or “coupled” to another feature or element, it can be directly connected, attached or coupled to the other feature or element or intervening features or elements may be present. In contrast, when a feature or element is referred to as being “directly connected”, “directly attached” or “directly coupled” to another feature or element, there are no intervening features or elements present. Although described or shown with respect to one embodiment, the features and elements so described or shown can apply to other embodiments. It will also be appreciated by those of skill in the art that references to a structure or feature that is disposed “adjacent” another feature may have portions that overlap or underlie the adjacent feature.

Terminology used herein is for the purpose of describing particular embodiments only and is not intended to be limiting of the invention. For example, as used herein, the singular forms “a”, “an” and “the” are intended to include the plural forms as well, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof. As used herein, the term “and/or” includes any and all combinations of one or more of the associated listed items and may be abbreviated as “/”.

Spatially relative terms, such as “under”, “below”, “lower”, “over”, “upper” and the like, may be used herein for ease of description to describe one element or feature's relationship to another element(s) or feature(s) as illustrated in the figures. It will be understood that the spatially relative terms are intended to encompass different orientations of the device in use or operation in addition to the orientation depicted in the figures. For example, if a device in the figures is inverted, elements described as “under” or “beneath” other elements or features would then be oriented “over” the other elements or features. Thus, the exemplary term “under” can encompass both an orientation of over and under. The device may be otherwise oriented (rotated 90 degrees or at other orientations) and the spatially relative descriptors used herein interpreted accordingly. Similarly, the terms “upwardly”, “downwardly”, “vertical”, “horizontal” and the like are used herein for the purpose of explanation only unless specifically indicated otherwise.

Although the terms “first” and “second” may be used herein to describe various features/elements (including steps), these features/elements should not be limited by these terms, unless the context indicates otherwise. These terms may be used to distinguish one feature/element from another feature/element. Thus, a first feature/element discussed below could be termed a second feature/element, and similarly, a second feature/element discussed below could be termed a first feature/element without departing from the teachings of the present invention.

Throughout this specification and the claims which follow, unless the context requires otherwise, the word “comprise”, and variations such as “comprises” and “comprising” means various components can be co-jointly employed in the methods and articles (e.g., compositions and apparatuses including device and methods). For example, the term “comprising” will be understood to imply the inclusion of any stated elements or steps but not the exclusion of any other elements or steps.

In general, any of the apparatuses and methods described herein should be understood to be inclusive, but all or a sub-set of the components and/or steps may alternatively be exclusive, and may be expressed as “consisting of” or alternatively “consisting essentially of” the various components, steps, sub-components or sub-steps.

As used herein in the specification and claims, including as used in the examples and unless otherwise expressly specified, all numbers may be read as if prefaced by the word “about” or “approximately,” even if the term does not expressly appear. The phrase “about” or “approximately” may be used when describing magnitude and/or position to indicate that the value and/or position described is within a reasonable expected range of values and/or positions. For example, a numeric value may have a value that is +/−0.1% of the stated value (or range of values), +/−1% of the stated value (or range of values), +/−2% of the stated value (or range of values), +/−5% of the stated value (or range of values), +/−10% of the stated value (or range of values), etc. Any numerical values given herein should also be understood to include about or approximately that value, unless the context indicates otherwise. For example, if the value “10” is disclosed, then “about 10” is also disclosed. Any numerical range recited herein is intended to include all sub-ranges subsumed therein. It is also understood that when a value is disclosed that “less than or equal to” the value, “greater than or equal to the value” and possible ranges between values are also disclosed, as appropriately understood by the skilled artisan. For example, if the value “X” is disclosed the “less than or equal to X” as well as “greater than or equal to X” (e.g., where X is a numerical value) is also disclosed. It is also understood that the throughout the application, data is provided in a number of different formats, and that this data, represents endpoints and starting points, and ranges for any combination of the data points. For example, if a particular data point “10” and a particular data point “15” are disclosed, it is understood that greater than, greater than or equal to, less than, less than or equal to, and equal to 10 and 15 are considered disclosed as well as between 10 and 15. It is also understood that each unit between two particular units are also disclosed. For example, if 10 and 15 are disclosed, then 11, 12, 13, and 14 are also disclosed.

Although various illustrative embodiments are described above, any of a number of changes may be made to various embodiments without departing from the scope of the invention as described by the claims. For example, the order in which various described method steps are performed may often be changed in alternative embodiments, and in other alternative embodiments one or more method steps may be skipped altogether. Optional features of various device and system embodiments may be included in some embodiments and not in others. Therefore, the foregoing description is provided primarily for exemplary purposes and should not be interpreted to limit the scope of the invention as it is set forth in the claims.

The examples and illustrations included herein show, by way of illustration and not of limitation, specific embodiments in which the subject matter may be practiced. As mentioned, other embodiments may be utilized and derived there from, such that structural and logical substitutions and changes may be made without departing from the scope of this disclosure. Such embodiments of the inventive subject matter may be referred to herein individually or collectively by the term “invention” merely for convenience and without intending to voluntarily limit the scope of this application to any single invention or inventive concept, if more than one is, in fact, disclosed. Thus, although specific embodiments have been illustrated and described herein, any arrangement calculated to achieve the same purpose may be substituted for the specific embodiments shown. This disclosure is intended to cover any and all adaptations or variations of various embodiments. Combinations of the above embodiments, and other embodiments not specifically described herein, will be apparent to those of skill in the art upon reviewing the above description.

1. A system for removing a clot material, the system comprising:
an aspiration line configured to couple to an aspiration catheter;
a clot capture chamber in fluid communication with the aspiration line and configured to capture the clot material;
a peristaltic pump apparatus coupled to the aspiration line;
a collection chamber coupled to the aspiration line and configured to collect blood aspirated through the aspiration line; and
a control for controlling operation of the peristaltic pump apparatus, wherein the control is configured to cause the peristaltic pump apparatus to apply aspiration to the aspiration line at a high flow rate when activated.

2. The system of claim 1, wherein the peristaltic pump apparatus is configured to apply aspiration at a high flow rate of 1.5 L/min or greater when activated by the control.

3. The system of claim 1, wherein the peristaltic pump apparatus is configured to apply aspiration at a high flow rate of 5 L/min or greater when activated by the control.

4. The system of claim 1, wherein the peristaltic pump apparatus comprises two or more peristaltic pump heads arranged in parallel.

5. The system of claim 4, wherein the two or more peristaltic pump heads arranged in parallel comprise a first pump having a high flow and low pressure and a second pump having a low flow and high pressure.

6. The system of claim 4, wherein the two or more peristaltic pump heads arranged in parallel are configured to have rollers that are out of phase with each other such that the rollers engage the tubing on the first pump at least 10 degrees of rotation before the rollers engage the tubing on the second pump.

7. The system of claim 6, wherein the rollers are out of phase such that the roller of a first peristaltic pump head fully engages peristaltic tubing within before any other peristaltic pump head begins engaging peristaltic tubing.

8. The system of claim 1, wherein the collection chamber is part of a blood return sub-system further comprising a blood return line configured to couple to a blood return inlet on the patient.

9. The system of claim 6, wherein the blood return sub-system comprises a second pump configured to drive fluid into the patient.

10. The system of claim 1, wherein the collection chamber comprises an expandable container.

11. The system of claim 1, wherein the collection chamber comprises one or more filters.

12. The system of claim 1, wherein the control is further configured to open a valve on aspiration line when the peristaltic pump apparatus is activated.

13. The system of claim 1, wherein the clot capture chamber is coupled to the aspiration line proximal to the peristaltic pump apparatus.

14. The system of claim 1, wherein the collection chamber is coupled to the aspiration line distal to the peristaltic pump apparatus.

15. The system of claim 1, wherein the control is configured to apply aspiration at a first, high, flow rate for a first time, then at a second, lower flow rate for a second time.

16. The system of claim 1, wherein the peristaltic pump apparatus comprises a vacuum augmented peristaltic pump.

17.-49. (canceled)

50. A method for the removing clot material from a vasculature of a patient, the method comprising:
positioning a catheter at least partially within the vasculature proximate to the clot material, wherein the catheter is configured to be fluidically coupled to a peristaltic pump apparatus, and wherein activating the peristaltic pump apparatus fluidically connects the peristaltic pump apparatus to the catheter and wherein de-activating the peristaltic pump apparatus fluidically disconnects the peristaltic pump apparatus from the catheter;
activating the peristaltic pump apparatus to generate negative pressure within the catheter to apply the negative pressure to generate a flow rate within the catheter of greater than about 5 L/min to aspirate at least a portion of the clot material into the catheter.

51. The method of claim 50, wherein the peristaltic pump apparatus comprises a vacuum augmented peristaltic pump, and further comprising generating a negative pressure around an outside of the tubing of the vacuum augmented peristaltic pump prior to or while activating the peristaltic pump apparatus.

52. The method of claim 50, wherein the peristaltic pump apparatus comprises two or more peristaltic pumps arranged in parallel.

53. The method of claim 52, wherein the two or more peristaltic pumps arranged in parallel comprise a first pump having a high flow and low pressure and a second pump having a low flow and high pressure.

54.-57. (canceled)

58. A system for removing clot material, the system comprising:
an aspiration catheter;
an aspiration line coupled to the aspiration catheter;
an on-demand vacuum source, wherein the on-demand vacuum source is in fluid communication with aspiration catheter through the aspiration line, and wherein the on-demand vacuum source is configured to generate aspiration by moving an actuator against a region of the aspiration line;
a valve between the aspiration catheter and the one-demand vacuum source; and
a control configured to open the valve when the on-demand vacuum source is activated and to close the valve when the on-demand vacuum source is deactivated so that vacuum is not stored in the system.

59. A system for removing clot material, the system comprising:
an aspiration catheter;
an aspiration line coupled to the aspiration catheter;
a peristaltic pump apparatus coupled to the aspiration line;
wherein the tubing within the peristaltic pump is partially comprised of Nitinol.

60. The system of claim 59 wherein the tubing is further comprised of a low durometer elastomer with a wall thickness of greater than 0.1 mm.

61. The system of claim 59 wherein the Nitinol is wrapped in a spiral pattern and integrated into the wall of the tubing.

62. The system of claim 57 wherein the Nitinol is a braided structure and is integrated into the wall of the tubing.

63. The system of claim 59 wherein the Nitinol is within the inner lumen of the tubing and wherein a portion of the Nitinol is in direct contact with the fluid within the tubing.

64.-72. (canceled)
